SEQUENCE LISTING (20 sequences)

SEQ ID NO: 1 - DNA, 22 bp, Artificial Sequence
  oligonucleotide
gagcacaagcttgccaacatcg

SEQ ID NO: 2 - DNA, 24 bp, Artificial Sequence
  oligonucleotide
ggaattccttaatatttacttttt

SEQ ID NO: 3 - DNA, 20 bp, Artificial Sequence
  oligonucleotide
agtagtctggacaaatattt

SEQ ID NO: 4 - DNA, 29 bp, Artificial Sequence
  oligonucleotide
ccggaattcttatctatttataatttcat

SEQ ID NO: 5 - DNA, 21 bp, Artificial Sequence
  oligonucleotide
gagtagtaagaaagactatat

SEQ ID NO: 6 - DNA, 27 bp, Artificial Sequence
  oligonucleotide
ggaattccttatctatttataatttca

SEQ ID NO: 7 - DNA, 25 bp, Artificial Sequence
  oligonucleotide
gagcaaatctgaaactacatcacat

SEQ ID NO: 8 - DNA, 27 bp, Artificial Sequence
  oligonucleotide
ggaattccttaattgttttgtgaatgc

SEQ ID NO: 9 - DNA, 510 bp, Staphylococcus aureus
atctatatagttaatgaataattaatgtacttttttttagttagtcattaaaataaattagtactaattactaaggagaataaaaaatgaaaattagaaaatctatacttgcgggaactttagcaatcgttttagcatcaccactagtaactaatctagataaaaatgaggcacaagctagcacaagcttgccaacatcgaatgaatatcaaaacgaaaagttagctaatgaattaaaatcgttattagatgaactaaatgttaatgaattagctactggaagtttaaacacttattataagcgaactataaaaatttcaggtcaaaaagcaatgtatgctcttaagtcaaaagactttaagaaaatgtcagaagcaaaatatcaacttcaaaagatttataacgaaattgacgaagcactaaaaagtaaatattaaaaaaaccacccgtaaaagggtggttttaattttctagataatataaaagtgttcataaataaaacagtatagg

SEQ ID NO: 10 - DNA, 510 bp, Staphylococcus aureus
atctatatagttaatgaataattaatgtacttttttttagttagtcattaaaataaattagtactaattactaaggagaataaaaaatgaaaattagaaaatctatacttgcgggaactttagcaatcgttttagcatcaccactagtaactaatctagataaaaatgaggcacaagctagcacaagcttgccaacatcgaatgaatatcaaaacgaaaagttagctaatgaattaaaatcgttattagatgaactaaatgttaatgaattagctactggaagtttaaacacttattataagcgaactataaaaatttcaggtctaaaagcaatgtatgctcttaagtcaaaagactttaagaaaatgtcagaagcaaaatatcaacttcaaaagatttataacgaaattgacgaagcactaaaaagtaaatattaaaaaaaccacccgtaaaagggtggttttaattttctagataatataaaagtgttcataaataaaacagtatagg

SEQ ID NO: 11 - DNA, 364 bp, Staphylococcus aureus
ggagagtttacaatgaaatttaaaaaatatatattaacaggaacattagcattacttttatcatcaactgggatagcaactatagaagggaataaagcagatgcaagtagtctggacaaatatttaactgaaagtcagtttcatgataaacgcatagcagaagaattaagaactttacttaacaaatcgaatgtatatgcattagctgcaggaagcttaaatccatattataaacgtacgattatgatgaatgaatatagagctaaagcggcacttaagaaaaatgatttcgtatcaatggctgatgctaaagttgcattagaaaaaatatacaaagaaattgatgaaattataaatagataat

SEQ ID NO: 12 - DNA, 364 bp, Staphylococcus aureus
ggagaatttacaatgaaatttaaaaaatatatagtagcaggaacattagcagtactattatcaacaacagcagtatcaacgttagatgggaataaagcagatgcaagtagtaagaaagactatataattcaaagtgagtttcatgataaacgaattgctgaagaattgaaatcattacttgatcaatcttatgtaaatgatttagctgcaggaagcttaaacccatactacaaacgtatgattatgatgaaccaatatagagcaaaagcagcactaaaaagtaataatttcgcaaaaatggctgaagctaaagttggattagaaaacatttacaaagaaattgatgaaattataaatagataat

SEQ ID NO: 13 - DNA, 384 bp, Staphylococcus aureus
ggagtaacaaagcatgacaacacaaatgaaaatcaaaacatatttagttgctggtattaaagcggcgctccttgatacgactggtattaaattagcaagcaaatctgaaactacatcacatacgtatcaacatcaagcgcttgtagatcaattacatgaattaatagcaaacactgacttaaataaattatcgtacctaaatttagatgcgtttcaaaaacgcgatattttagctgcgcactatattgcaaaatccgctatacgcactaaaaatttggatcaaatgactaaagcgaaacaaagattagaaagtatttacaagattagaaagtatttacrattcaatttctaaccctttgcattcacaaaacaattaataattca

SEQ ID NO: 14 - DNA, 384 bp, Staphylococcus aureus
ggagtaacaaagcatgacaacacaaatgaaaatcaaaacatatttagttgctggtattaaagcggcgctccttgatacgactggtattaaattagcaagcaaatctgaaactacatcacatacgtatcaacatcaagcgcttgtagatcaattacatgaattaatagcaaacactgacttaaataaattatcgtacctaaatttagatgcgtttcaaaaacgcgatattttagctgcgcactatattgcaaaatccgctatacgcactaaaaatttggatcaaatgactaaagcgaaacatagattagaaagtatttacgagattagaaagtatttacrattcaatttctaaccctttgcattcacaaaacaattaataattca

SEQ ID NO: 15 - protein, 116 aa, Staphylococcus aureus
MKIRKSILAGTLAIVLASPLVTNLDKNEAQASTSLPTSNEYQNEKLANELKSLLDELNVNELATGSLNTYYKRTIKISGQKAMYALKSKDFKKMSEAKYQLQKIYNEIDEALKSKY

SEQ ID NO: 16 - protein, 116 aa, Staphylococcus aureus
MKIRKSILAGTLAIVLASPLVTNLDKNEAQASTSLPTSNEYQNEKLANELKSLLDELNVNELATGSLNTYYKRTIKISGLKAMYALKSKDFKKMSEAKYQLQKIYNEIDEALKSKY

SEQ ID NO: 17 - protein, 116 aa, Staphylococcus aureus
MKFKKYILTGTLALLLSSTGIATIEGNKADASSLDKYLTESQFHDKRIAEELRTLLNKSNVYALAAGSLNPYYKRTIMMNEYRAKAALKKNDFVSMADAKVALEKIYKEIDEIINR

SEQ ID NO: 18 - protein, 116 aa, Staphylococcus aureus
MKFKKYIVAGTLAVLLSTTAVSTLDGNKADASSKKDYIIQSEFHDKRIAEELKSLLDQSYVNDLAAGSLNPYYKRMIMMNQYRAKAALKSNNFAKMAEAKVGLENIYKEIDEIINR

SEQ ID NO: 19 - protein, 114 aa, Staphylococcus aureus
MTTQMKIKTYLVAGIKAALLDTTGIKLASKSETTSHTYQHQALVDQLHELIANTDLNKLSYLNLDAFQKRDILAAHYIAKSAIRTKNLDQMTKAKQRLESIYNSISNPLHSQNN

SEQ ID NO: 20 - protein, 114 aa, Staphylococcus aureus
MTTQMKIKTYLVAGIKAALLDTTGIKLASKSETTSHTYQHQALVDQLHELIANTDLNKLSYLNLDAFQKRDILAAHYIAKSAIRTKNLDQMTKAKHRLESIYDSISNPLHSQNN

(19) United States
(12) Patent Application Publication
(10) Pub. No.: US 2009/0246175 A1
(43) Pub. Date: Oct. 1, 2009

(54) THERAPEUTIC USE OF SCIN, A STAPHYLOCOCCAL COMPLEMENT INHIBITOR IN INFLAMMATORY DISEASES

(75) Inventors: Suzan H. M. Rooijakkers, Den Bosch (NL); Willem Jan Bastiaan Van Wamel, Utrecht (NL); Cornelis P. M. Van Kessel, Bunnik (NL); Johannes A. G. Van Strijp, Odijk (NL)

Correspondence Address: BOZICEVIC, FIELD & FRANCIS LLP, 1900 UNIVERSITY AVENUE, SUITE 200, EAST PALO ALTO, CA, 94303, US

(73) Assignee: UMC Utrecht Holding B.V., Utrecht (NL)

(21) Appl. No.: 11/909,915

(22) PCT Filed: Apr. 3, 2006
(86) PCT No.: PCT/EP2006/003449

§ 371 Date: Jul. 21, 2008

(30) Foreign Application Priority Data
Apr. 1, 2005 (EP) 05075765.7

(51) Int. Cl. A61K 35/00; A61K 38/16

(52) U.S. Cl. 424/93.2; 514/12

(57) ABSTRACT

The present invention relates to the use of the staphylococcal complement inhibitor (SCIN) or a homologue thereof, or a derivative or a fragment of SCIN or the SCIN homologue for the preparation of a medicament for intervening with C3 and C5 convertases. The medicament is in particular intended for inhibiting activation of the classical and/or the alternative pathway of complement. More in particular, the medicament is for treating indications that involve complement activation via the classical and/or alternative pathway.

The present invention relates to the use of a polypeptide having complement inhibitory activity for the inhibition of all known complement activation pathways. The polypeptide, called herein Staphylococcus Complement INhibitor (SCIN), can be used in the treatment of inflammatory reactions. In addition to polypeptides, derivatives and fragments thereof can be used, such as peptides and non-(poly)peptides.

Complement is the complex network of over 20 serum proteins that are part of our innate immune system. Complement acts by itself (through lysis of microbes) or in conjunction with other components of the innate immune system (e.g. phagocytosis). Our innate immune system is mainly involved in protecting the body against foreign invaders (e.g. bacteria, viruses, fungi, and also cancer cells). The most important cells of the innate immune system are dendritic cells, monocytes/macrophages and neutrophils. Next to that, our innate immune system contains a large variety of soluble factors such as acute phase proteins, antimicrobial peptides, peptidases, parts of the clotting cascade and the complement system. Killing and removal of invaders is mostly done by monocytes and neutrophils, by direct recognition of the invaders or with the help of specific antibodies and/or the complement system (opsonization).

Cells of the innate system react in a relatively aggressive way. Since they are part of the body's first line of defense, their most important task is to kill and remove the invading agent as quickly as possible. This is accomplished through very aggressive substances (e.g. free radicals and enzymes) that are not only lethal to the invader, but also cause damage to host cells in the vicinity. Substances from these damaged cells and the locally activated cells from the innate system itself will further attract increasing numbers of neutrophils and monocytes, causing further local inflammation.

In most cases, such an aggressive and damaging inflammatory reaction, caused by over-activated neutrophils, is unnecessary. In some cases this inflammatory response is responsible for serious, sometimes lethal disorders and includes conditions like Adult Respiratory Distress Syndrome (ARDS), severe tissue damage following thrombotic events such as heart attacks and stroke, inflammatory bowel diseases and rheumatoid arthritis.

The inflammation will subside once all the invaders have been killed and removed, together with the various cells killed in the process. Healing of the wound, caused by the inflammatory response, can then begin. Although there is some overlap in function, the main task of neutrophils is to attack the invaders and the main task of monocytes is to remove the debris resulting from this attack. In addition, neutrophils have another peaceful task in assisting the wound healing process.

When bacteria have invaded the body, substances of microbial origin activate the complement system directly or via pre-existing antibodies. The first molecule involved in antibody-mediated complement activation is C1q followed by the activation of C1r and C1s. A parallel pathway does not need specific antibodies, because it directly recognizes microbial surface structures. H-ficolin, L-ficolin, M-ficolin or Mannose Binding Lectin (MBL) recognize microbes and through activation of specific MBL Associated Serine Proteases (MASP-2) the rest of the complement system is activated.

In both events activation proceeds through C4 and C2 and the central molecule of the complement system C3 is activated. This leads to more C3 deposition via the alternative pathway (factors B, D, H, I, P). C3 once converted into C3b, C3bi or even C3d is the most important opsonin, it mediates uptake of microbes by phagocytes, and importantly also activates these phagocytes in the process. Next to this phagocyte directed action, the complement system can proceed from C3 via C5, C6, C7, C8, and C9 to lysis of the tumor cell, virus infected cell, gram negative bacterium, or during unwanted inflammatory events, one of the healthy cells of our body.

Normally our cells are protected from unwanted complement attack by a variety of mechanisms (C1INH, C4 bp, CR1, MCP, DAF, H, I, P, CD59) but in cases of disturbance or extremely high local activation, direct complement mediated damage can still occur. Furthermore, in the process of complement activation, the formation of opsonins and membrane attack is parallelled by the formation of very strong inflammatory small molecules (C5a, C3a). These substances directly activate phagocytes and other cells (via C5a and C3a receptors) in a very efficient way, resulting in damage to microbes or healthy cells. The interaction with different cell types also gives rise to the production of other chemokines (like interleukin-8, IL-8): substances that can activate and attract cells from the blood vessels (the migration process). Neutrophils interact with these substances, because they have receptors for these substances on the outside of their cell membrane. An overview of the components of the complement system is given in Table 1.

TABLE 1
PROTEINS INVOLVED IN THE COMPLEMENT CASCADE

| | |
|---|---|
| Binding to Ag:Ab complexes: | C1q |
| Direct recognition of | MBL, Ficolin-H, L-Ficolin, |
| microbial surface structures: | M-Ficolin |
| Activating Enzymes: | C1r, C1s, C2a, Bb, factor D, |
| | MASP1, 2, 3, C3 convertase (C4b2a, |
| | C3bBb), C5 convertase (C3bC4b2a, |
| | C3bC3bBb) |
| Membrane-binding opsonins: | C4b, C3b |
| Mediators of inflammation: | C5a, C3a, C4a |
| Membrane attack: | C5b, C6, C7, C8, C9 |
| Complement Receptors: | CR1, CR2, CR3, CR4, C1qR, M-Ficolin |
| Complement-regulatory | C1INH, C4bp, CR1, MCP, DAF, H, I, |
| proteins: | P, CD59 |

*Adapted from Janeway & Travers Immunobiology, 1996; Current Biology Ltd/Garland Publishing Inc.

Activated neutrophils can easily migrate from blood vessels. This is because the chemokines and microbial products will have increased the permeability of the vessels and stimulated the endothelial cells of the vessel walls to express certain adhesion molecules. Neutrophils express selectins and integrins (e.g. CD11b/CD18) that bind to these adhesion molecules. This process is called priming. Once the neutrophil has adhered to the endothelial cells, it is able to migrate through the cells, under the guidance of chemokines, towards the site of infection, where the concentration of these substances is at its highest.

These substances also activate neutrophils to produce a range of other molecules, some of which attract more neutrophils (and subsequently monocytes), but, mostly, they are responsible for destroying the invading bacteria. Some of these substances (e.g. free radicals, enzymes that break down proteins (proteases) and cell membranes (lipases)) are so reactive and non-specific that cells from the surrounding tissue (and the neutrophils themselves) are destroyed, causing tissue damage. This damage is exacerbated by the presence of blood-derived fluid, which has transgressed the leaky vessel wall and is responsible for the swelling that always accompanies inflammation (called edema). The pressure build up caused by this excess fluid results in further cell damage and death.

The onset of an inflammatory reaction does not have to be of microbial origin per se. Tissue damage in general, by oxygen deprivation, pH changes, salt disbalance or physical damage can start inflammatory reactions. In many cases the key event is the activation of the complement system. In autoimmune diseases, the presence of auto-antibodies gives rise to the activation of the classical pathway of complement. In almost all other events the activation of complement is via the lectin pathway or via the alternative pathway (cf. Jordan et al., Circulation. 2001, 104(12):1413-8; Collard et al., Am J Pathol. 2001, 159:1045-54; Roos et al., J Immunol. 2001, 167:2861-8; Collard et al., Am J Pathol. 2000, 156:1549-56; Collard et al., Mol Immunol. 1999, 36:941-8).

Later in the inflammatory process, monocytes migrate to the scene and become activated. Besides their role in removing bacteria and cell debris, they also produce substances such as tumor necrosis factor (TNF) and IL-8, which in turn attract more activated neutrophils, causing further local damage. TNF also has a direct stimulatory effect on neutrophils. Once all the invaders have been removed, the inflammatory response will subside and the area will be cleared of the remaining “casualties”. Then the process of wound healing starts. Although it is known that neutrophils play a pivotal role in wound healing, it is not clear which neutrophil-derived substances are involved and how the neutrophils are active in healing without being aggressive to the surrounding tissue. In general, damaged tissue will be replaced by scar tissue formed mainly of fibroblasts and collagen.

When inflammation occurs in areas of the body with an important function, like tissues formed from heart muscle cells, brain cells or lung alveolar cells, normal function will be compromised by the resulting scar formation, causing serious conditions like heart failure, paralysis and emphysema. To minimize the debilitating consequences of these conditions, it is important to “dampen” the inflammatory reaction as quickly as possible.

Intervention to control the acute early phase inflammatory response presents an opportunity to improve the prognosis for a wide range of patients whose symptoms can be traced back to such an event. Such an approach has been advocated for many acute and chronic inflammation based diseases and shown to have potential based on findings from relevant disease models. Classical anti-inflammatory drugs such as steroids and Non Steroid Anti-Inflammatory Drugs (NSAIDS) do not have the ideal profile of action, either in terms of efficacy or safety. Steroids affect the “wrong” cell type (monocytes) and their dampening effects are easily bypassed. NSAIDS generally show a relatively mild effect partly because they intervene at a late stage in the inflammatory process. Both classes of drugs produce a range of undesirable side effects resulting from other aspects of their pharmacological activity.

Several drugs under early development only interfere with late mediators in the route to neutrophil activation (e.g. C5 convertase inhibitors, antibodies against C5a, C5a-receptor blocking drugs, antibodies against integrins (like CD11b/CD18) and L-selectin on neutrophils and antibodies against adhesion molecules (like ICAM-1 and E-selectin) on endothelial cells).

Antibodies against TNF and IL-8 have effects in chronic inflammation, but only marginal effects in acute inflammation, because of the minimal role monocytes (which are mainly responsible for these substances' production) play in the acute phase and because they react even later in the inflammation cascade. In many cases it would be extremely desirable to stop the inflammatory cascade in an early-as-possible-phase. This is also true because this cascade is not linear but branches off at different stages causing redundancy in the later steps.

Sometimes, the cause of the acute inflammation cannot be removed and the inflammation becomes chronic. With the exception of tuberculosis, chronic hepatitis and certain other conditions, this is seldom the case with infections. However, chronic inflammation can also be caused by stimuli other than bacteria, such as auto-immune reactions. Research has shown that in chronic inflammation the role of monocytes is much more prominent, and that neutrophil migration and activation, monocyte migration and activation, tissue damage, removal of dead cells and wound healing are all going on at the same time.

This complex cascade of interactions between cells and many different cytokines and chemokines has been the subject of intensive research for many years. It was believed that monocytes and their products were the most important elements that needed to be inhibited to dampen chronic inflammation. This explains why steroids, which specifically interact with monocytes, are generally more effective in chronic as opposed to acute inflammation. Long-term treatment with steroids however, is not a desirable option, because severe and unacceptable side effects can occur at the doses required to produce a clinical effect.

Newer treatments using antibodies against TNF or IL-8 have shown good results, and were initially seen as proof of the major role monocytes were thought to play in chronic inflammation. Recent research casts doubts on an exclusive role for monocytes in inflammation and points to a critical role for neutrophils, which are now seen to represent better targets for therapeutic intervention.

The complement system as a major component of the innate immune system is thus involved in initiation of the adaptive immune response. The complement system acts via three separate pathways that differ in their mode of recognition but converge in the generation of C3 convertases that cleave the central component C3. The C3 convertases mediate virtually all biological activities of the complement system. Cleavage of C3 results in the release of the anaphylactic agent C3a and in the covalent attachment of C3b to the microbial surface, a process called opsonization. Opsonization is a crucial step in immunity, because surface-bound C3b and its degradation products facilitate the recognition of foreign substances by phagocytic cells. Later on, the C3 convertase is changed into a C5 convertase by inclusion of another C3b molecule to the C3 convertase. This C5 convertase cleaves C5 resulting in release of the potent chemo-attractant C5a and formation of the lytic C5b-9 complex.

The assembly of C3 convertases proceeds through sequential steps involving protein-protein interactions and proteolytic cleavages by highly specific serine proteases. In the classical pathway (CP) and lectin pathway (LP) C3 convertase (C4b2a) is generated through cleavage of C4 and C2 by C1s or Mannan Binding Lectin (MBL)-associated Serine Protease-2 (MASP-2). The protease C1s circulates in complex with the recognition molecule of the CP, C1q, which binds antigen-bound IgG or IgM. MASP-2 is in complex with MBL or ficolin that recognize conserved sugars patterns on microbes. At first, C1s and MASP-2 catalyze the covalent binding of C4b to the surface of the activator. C2 binds to surface-bound C4b and as such is cleaved by C1s or MASP-2, generating C4b2a.

The alternative pathway (AP) lacks a specific recognition molecule. In this pathway, the assembly of C3 convertases is initiated by covalent attachment of C3b to the activator surface. In the next step, factor B (fB) binds to surface-bound C3b and is subsequently cleaved by factor D (fD), generating C3bBb. Both C4b2a and C3bBb are subsequently transformed into a C5 convertase by the binding of an additional C3b molecule.

The proteins that form the CP and LP C3 convertases are structurally and functionally similar to those forming the AP convertase. Both C4 and C2 show significant sequence similarity to C3 and fB respectively; they are derived from common ancestors. Although C3 convertase activity resides in one molecule (C2a or Bb), the capacity to cleave C3 is acquired only through complex formation. Amplification of convertases is regulated in several ways including the intrinsic decay of the inherently labile C4b2a and C3bBb complexes. Decay of C3bBb can be delayed by binding of the glycoprotein properdin. Dissociated C2a and Bb are inactive and cannot re-associate with C4b and C3b to form new convertases.

Staphylococcus aureus (S. aureus) is an important human pathogen that causes a wide range of diseases by production of various cell wall-associated and excreted proteins. A number of these proteins contribute to the escape of the bacterium from the immune system. In the research that led to the present invention a new S. aureus Pathogenicity Island 5 (SaPI5) was identified encoding four excreted proteins of which three interact with the human immune system: CHIPS is a Chemotaxis Inhibitory Protein of Staphylococci, Staphylokinase (SAK) is anti-phagocytic and able to inhibit bactericidal activity of alpha-defensins and Staphylococcal Enterotoxin A (SEA) is known as a superantigen but also involved in down-modulation of chemokine receptors on monocytes.

All the proteins on this pathogenicity island were shown to be human specific. The human specificity of CHIPS was shown by a 30-fold difference in activity towards human cells as compared to cells from mouse, rat, dog, guinea pig, pig and rabbit. Also, the response of mouse T cells to the superantigen SEA requires 1000-fold higher concentrations compared to human T cells. Staphylokinase only activates plasminogen of humans, dogs and baboons.

SaPI5 is transferred among staphylococci by bacteriophages that specifically incorporate into the β-hemolysin gene (hlb). Ninety percent of clinical and laboratory S. aureus strains carry SaPI5 and seven different types were identified.

The fourth gene on SaPI5 was identified and characterized as a staphylococcal lectin pathway inhibitor (LPI), an excreted 9.8 kDa protein, and is described in WO2005/005630. In this publication it is disclosed that LPI specifically inhibits the lectin pathway of complement activation.

In the research that led to the present invention it was now found that LPI is not specific for the lectin pathway but blocks all complement pathways by specific interaction with C4b2a and C3bBb. It binds and stabilizes C3/C5 convertases, interfering with C3b deposition via the classical, lectin and alternative complement pathway. This leads to a dramatic decrease in phagocytosis and killing of Staphylococcus aureus by human neutrophils. The polypeptide was thus renamed Staphylococcal Complement Inhibitor (SCIN).

As a highly active and small soluble protein that acts exclusively on surfaces, SCIN is a promising anti-inflammatory molecule.

The accession numbers for SCIN are gi|14247715|dbj|BAB58104.1| and gi|13701735|dbj|BAB43028.1|. The accession numbers for SCIN homologues in S. aureus are SCIN-B: gi|13700958|dbj|BAB42254.11, SCIN-C: gnl|sanger 159288|Staphylococcus (from the Sanger database (S. aureus 252, (MRSA 16) and ORF-D: gi/21203369|dbj|BAB94070.1|.

The present invention thus relates to the use of SCIN or a homologue thereof, or a derivate or a fragment of SCIN or the SCIN homologue for the preparation of a medicament for intervening with C3 and C5 convertases. More specifically the invention relates to the use of SCIN or a homologue thereof, or a derivate or a fragment of SCIN or the SCIN homologue for the preparation of a medicament for inhibiting activation of the classical and alternative pathway of complement for treating indications that involve complement activation via the classical or alternative pathway, in particular for treating inflammation.

In the application the definition of SCIN activity is as follows:

SCIN prevents activation of the classical, lectin and alternative pathway of complement specifically by intervening with C3 and C5 convertases. The binding of SCIN to C3 and C5 convertases results in

a) inactivation of the convertases,

b) stabilization of the convertases and

c) diminished formation of new convertases.

Through this interaction with complement convertases, SCIN blocks all biologically important actions of the complement cascade activation: C3b/iC3b deposition, C3a/C5a production and the generation of C5b-9.

The polypeptide having SCIN activity for use according to the invention is encoded by a nucleic acid molecule (the gene for SCIN is designated scn), comprising a nucleotide sequence, said nucleotide sequence corresponding to a sequence being selected from the group consisting of:

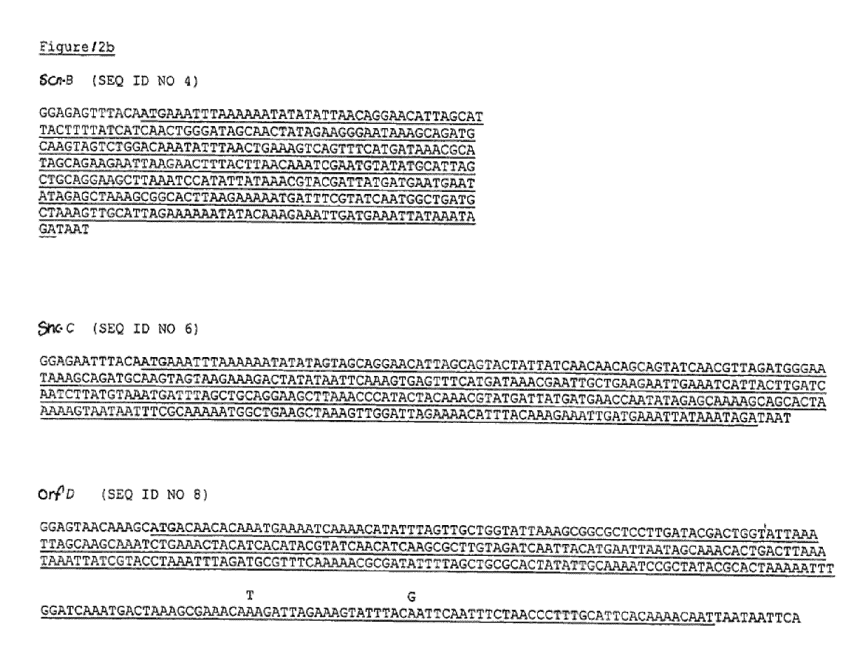

a) a nucleotide sequence comprising at least part of the sequence of scn, scn-B or scn-C as depicted in FIG. 12a or 12b (SEQ ID NO:2, SEQ ID NO:4, SEQ ID NO:6);

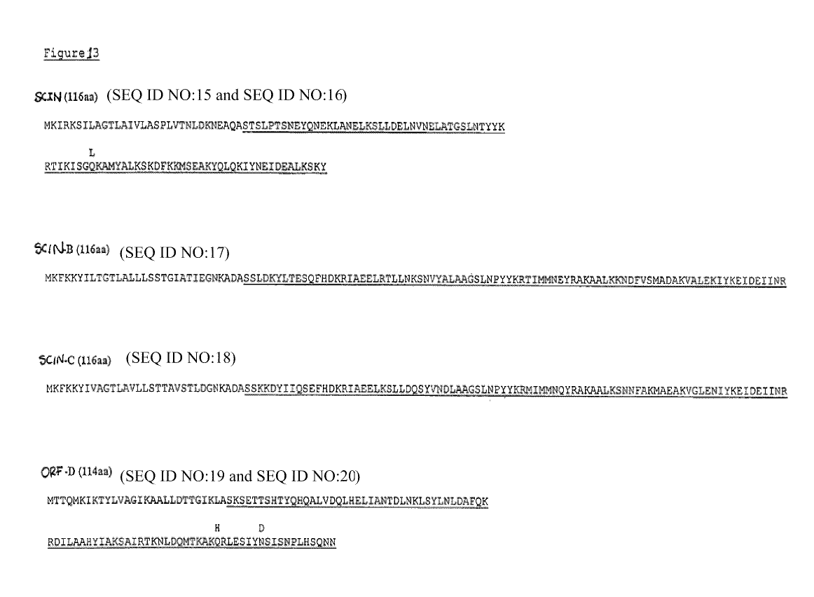

b) nucleotide sequences encoding a (poly)peptide having SCIN activity and having one of the amino acid sequences depicted in FIG. 13 identified as SEQ ID NO:3, SEQ ID NO:5 or SEQ ID NO:7;

c) nucleotide sequences encoding a (poly)peptide having SCIN activity and having a portion of the amino acid sequences depicted in FIG. 13 identified as SEQ ID NO:3, SEQ ID NO:5 or SEQ ID NO:7;

d) nucleotide sequences being at least 40% identical to any one of the nucleotide sequences a), b) or c);

e) nucleotide sequences hybridizing at stringent conditions with any one of the nucleotide sequences a), b), c) or d); and

f) nucleotide sequences complementary to any of the nucleotide sequences a), b), c), d) or e).

The complete genome of Staphylococcus aureus was sequenced and can be found in the regular international databases like GenBank, RefSeq, and PDB. The accession numbers for the SCIN proteins are gi|14247715|dbj|BAB58104.1| and gi|13701735|dbj|BAB43028.1|. The SCIN gene encodes a protein of 116 amino acids which shares 35-45% homology with other Staphylococcus aureus proteins of the same size, SCIN-B (gi|13700958|dbj|BAB42254.1|; gi|14246929|dbj|BAB57321.1|), and SCIN-C (gnl|Sanger_{—}159288|Staphylococcus (from the Sanger database (S. aureus 252, (MRSA 16)).

Regarding the term “identical” under d) above it should be noted that identicity and homology are used interchangeably. It should furthermore be noted that for gapped alignments, statistical parameters can be estimated using the Smith-Waterman algorithm that produces optimal alignment scores. Homologues of the SCIN nucleic acid sequence or protein sequence are defined by a Gap Open Penalty of at least 12 and a Gap Expression Penalty of at least 1.

The sequence as given in FIG. 12a (SEQ ID NO:2) is one embodiment of the DNA sequence encoding the polypeptide for use according to the invention. It comprises a promoter region from nucleotides 1 to 86, a leader peptide sequence from nucleotides 87 to 179, the coding region for the (poly)peptide having SCIN activity from nucleotide 180 to 434, as well as a 3′ untranslated region from nucleotides 435 to 510.

The presented gene for SCIN of FIG. 12a or any nucleic acid derived therefrom may for example be operably linked to the trc expression system (Brosius et al., Gene 27: 161-172 (1984)). Many other suitable expression control sequences and methods of expressing recombinant proteins are known (F. M. Ausubel et al., Current Protocols in Molecular Biology, John Wiley and Sons, Inc., New York, N.Y.).

The nucleotide sequence as given in FIG. 12a also contains a leader peptide sequence. The coding region of the mature protein corresponds to nucleotides 180 to 434 of FIG. 12a. Other leader sequences can be used. Or the leader sequence may be omitted entirely, depending on the host cell in which the sequence is to be expressed.

The amino acid sequence in FIG. 13 (SCIN) (SEQ ID NO:3) is deduced from the DNA sequence in FIG. 12a. In a further embodiment of the invention the nucleic acid molecule thus may have a nucleotide sequence that corresponds to all degenerate variants of the SCIN gene, the SCIN-B gene or the SCIN-C gene.

The invention furthermore relates to the use of (poly)peptides that do not have the complete sequence of SCIN (SEQ ID NO:3), SCIN-B (SEQ ID NO:5) or SCIN-C (SEQ ID NO:7) from FIG. 13 but one or more functional portions thereof that in themselves or together constitute a biologically active (poly)peptide having SCIN activity. “A portion” as used herein does not exclude the possibility that a (poly)peptide comprises more than one portion and should thus be interpreted as “at least one”. Such portions may vary in size from the complete amino acid sequence minus one amino acid to peptides of at least 2, preferably at least 5 amino acids. In case the active part of the protein lies in two or more portions of the complete amino acid sequence, the invention also relates to nucleic acid sequences encoding these separate portions in a manner that leads to a peptide configuration that retains the biological activity. In practice this can for example mean that spacer sequences are to be incorporated in between biologically active portions to lead to a biologically active conformation.

Thus, when reference is made to “at least part of the sequence” this means not only the three parts described above (i.e. for SCIN: nucleotides 1-434, 87-434 and 180-434) but also other fragments of the gene or combinations thereof provided that they still encode a (poly)peptide having SCIN activity.

In a further embodiment thereof, the invention thus relates to the use of a polypeptide encoded by an isolated nucleic acid molecule which consists of the coding region of one or more portions of the amino acid sequence SCIN (SEQ ID NO:3), SCIN-B (SEQ ID NO:5) or SCIN-C (SEQ ID NO:7) from FIG. 13, wherein one portion of the amino acid sequence constitutes alone or with other portions of the amino acid sequence the region(s) of the (poly)peptide having SCIN activity that lead to biological activity.

The present invention is not limited to the use of polypeptides encoded by nucleic acid molecules having the exact same sequence as the sequence SCIN (SEQ ID NO:2), SCIN-B (SEQ ID NO:4) or SCIN-C (SEQ ID NO:6) depicted in FIGS. 12a and 12b or the above described variants thereof. Therefore, the invention relates to (poly)peptides encoded by nucleic acid molecules having a nucleotide sequence which is at least one of 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 91%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical or homologous to any one of the nucleotide sequences as defined under a), b) or c) above. Homology is to be determined over the entire length of the homologous sequence.

It was found that SCIN is less than 48% homologous to proteins and peptides known to date. The use of proteins and peptides that show at least 40% amino acid homology to the SCIN protein and have SCIN activity is thus also part of this invention.

The invention further relates to the use of polypeptides encoded by nucleic acid molecules having a nucleotide sequence hybridizing under stringent conditions with a nucleic acid molecule corresponding with the nucleotide sequence SCIN (SEQ ID NO:2), SCIN-B (SEQ ID NO:4) or SCIN-C (SEQ ID NO:6) given in FIG. 12a or 12b or degenerate sequences thereof, which encode an amino acid sequence SCIN (SEQ ID NO:3), SCIN-B (SEQ ID NO:5) or SCIN-C (SEQ ID NO:7) as given in FIG. 13. Stringent conditions are constituted by overnight hybridization at 42° C. in 5×SSC (SSC=150 mM NaCl, 15 mM trisodium citrate) and washing at 65° C. at 0.1×SSC. In addition to 5×SSC the hybridization solution may comprise 50% formamide, 50 mM sodium phosphate (pH 7.6), 5×Denhardt's solution, 10% dextran sulphate and 20 mg/ml denatured sheared salmon sperm DNA.

The use of the invention is also not limited to use of the (poly)peptide having SCIN activity encoded by the complete gene, but also relates to the use of fragments, derivatives and analogues thereof encoded by smaller nucleic acid molecules. “Fragments” are intended to encompass all parts of the (poly)peptide that retain its biological activity. “Fragments” can consist of one sequence of consecutive amino acids or of more than one of such sequences. “Derivatives” are the complete (poly)peptide having SCIN activity or fragments thereof that are modified in some way. Examples of modifications will follow herein below. “Analogues” are similar (poly)peptides having SCIN activity isolated from other organisms, in particular other pathogenic organisms.

All of the above categories have one thing in common, namely that they have “SCIN activity”. SCIN activity can be measured by any assay that shows inhibition of complement activation. Examples of such assays include deposition of C3b on bacteria, generation of C5a/C3a, complement deposition in ELISA format, phagocytosis, AP50 and others.

Therefore, for the present application, the term “(poly)peptides having SCIN activity” is intended to include the original SCIN, SCIN-B and SCIN-C proteins and their homologues in isolated or recombinant form, and other (poly)peptides, fragments, derivatives and analogues that exhibit SCIN activity.

The polypeptides having SCIN activity for use in the invention can be prepared as described for LPI in WO2005/005630.

The isolated nucleic acid molecule that encodes a (poly)peptide for use according to the invention may be DNA, RNA or cDNA.

The (poly)peptides having SCIN activity that are used according to the invention also include (poly)peptides characterized by amino acid sequences into which modifications are naturally provided or deliberately engineered. Modifications in the (poly)peptide or DNA sequences encoding the polypeptides can be made by those skilled in the art using known conventional techniques. Modifications of interest in the SCIN active (poly)peptide sequences may include replacement, insertion or deletion of selected amino acid residues in the coding sequence.

The functional activity of SCIN, the (poly)peptides, their fragments, derivatives and analogues can be assayed by various methods. Al methods that measure complement activation at one of its steps can be used as a readout. Thus, C2, fB, C5b-9 or C3 fragments-deposition (by flow cytometry or ELISA or immunoblotting), AP50 measurements using erythrocyte lysis, measurement of MAC complex or soluble split products of the complement cascade (ELISA or functional assays) and phagocytosis are all suitable candidates for measuring SCIN activity.

The use of isolated (poly)peptides having SCIN activity according to the invention may involve treating, preventing or ameliorating inflammatory conditions that are involved in many diseases and disorders, such as those listed in Table 2.

According to a further aspect thereof, the use of the invention may comprise diagnosis, prophylaxis or therapy, in particular the treatment of acute and chronic inflammation reactions, such as those listed in Table 2.

TABLE 2
Diseases caused by inflammatory reactions, involving complement
activation and/or neutrophil and or monocyte involvement.

acute reactive arthritis
acute transplant rejection
adult respiratory distress syndrome (ARDS)
alcoholic hepatitis
allergic rhinitis
allotransplantation
Alzheimer's disease
arteriosclerosis
arthus reaction
asthma
atherosclerosis
atopic dermatitis
bacterial meningitis
bacterial pneumonia
brain tumour
bronchogenic carcinoma
bullos pemphigoid
burn injuries
burns
cardiopulmonary bypass
cardiovascular diseases
chronic bronchitis
chronic lymph leukemia
chronic obstructive pulmonary disease (COPD)
contact dermatitis
Crohn's disease
cutaneous T-cell lymphoma
cystic fibrosis
dermatoses
diseases of the central nervous system
endometriosis
experimental allergic encephalomyelitis (EAE)
experimental allergic neuritis (EAN)
Forssman shock
frost bite
gastric carcinoma
gastrointestinal diseases
genitourinary diseases
glomerulonephritis
gout
haemolytic anemia
Heliobacter pylori gastritis
hemodialysis
hereditary angioedema
hypersensitivity pneumonia
idiopathic pulmonary fibrosis
immune complex (IC)-induced vasculitis
ischaemic shock
ischaemia-reperfusion episodes
ischemia-reperfusion injuries
joint diseases
(large) vessel surgery
metal fume fever
multiple sclerosis
multiple system organ failure
myasthenia gravis
Mycobacterium tuberculosis infection
myocardial infarction
pancreatitis
peritonitis
pleural emphesema
post- cardiopulmonary bypass (CBP) inflammation
psoriasis
repetitive strain injury (RSI)
respiratory diseases
rheumatoid arthritis
sepsis
septic shock
sinusitis
skin diseases
stroke
systemic lupus erythematosus (SLE)
transplantation
(traumatic) brain injury
Trichomonas vaginalis infection
ulcerative colitis
urinary tract infection
vascular leak syndrome
vasculitis
viral hepatitis
viral meningitis
viral respiratory tract infection
xenotransplantation
* Support for the therapeutical usefulness of the (poly)peptides of the invention for treatment of these diseases can be found in the following references: For ARDS: Demling R H (1995). The modern version of adult respiratory distress syndrome. Ann. Rev. Med. 46: 193-202; and Fujishima S, Aikawa N 1995 Neutrophil mediated tissue injury and its modulation. Intensive Care Med 21: 277-285; For severe infections (meningitis): Tunkel A R and Scheld W M (1993). Pathogenesis and pathophysiology of bacterial meningitis. Clin. Microbiol. Rev. 6: 118. For injury after ischaemia/reperfusion: Helier T, et al. (1999). Selection of a C5a receptor antagonist from phage libraries attenuating the inflammatory response in immune complex disease and ischemia/reperfusion injury. J. Immunol. 163: 985-994. For rheumatoid arthritis: Edwards S W and Hallett M B (1997). Seeing the wood for the trees: the forgotten role of neutrophils in rheumatoid arthritis. Immunology Today 18: 320-324; and Pillinger M H, Abramson S B (1995). The neutrophil in rheumatoid arthritis. Rheum. Dis. Clin. North Am. 1995 21: 691-714. For myocardial infarction: Byrne J G, Smith W J, Murphy M P, Couper G S, Appleyard R F, Cohn L H (1992). Complete prevention of myocardial stunning, contracture, low reflow, and edema after heart transplantation by blocking neutrophil adhesion molecules during reperfusion. J. Thorac. Cardiovasc. Surg. 104: 1589-96. For COPD: Cox G (1998). The role of neutrophils in inflammation. Can. Respir. J. 5 Suppl A: 37A-40A; and Hiemstra P S, van Wetering S, Stolk J (1998). Neutrophil serine proteinases and defensins in chronic obstructive pulmonary disease: effects on pulmonary epithelium. Eur. Respir. J. 12: 1200-1208. For stroke: Barone F C, Feuerstein G Z (1999). Inflammatory mediators and stroke: new opportunities for novel therapeutics. J. Cereb. Blood Flow Metab. 19: 819-834; and Jean W C, Spellman S R, Nussbaum E S, Low W C (1998). Reperfusion injury after focal cerebral ischemia: the role of inflammation and the therapeutic horizon. Neurosurgery 43: 1382-1396. For meningitis: Tuomanen E I (1996). Molecular and cellular mechanisms of pneumococcal meningitis. Ann. N. Y. Acad. Sci. 797: 42 52. For all directly complement related diseases: Adapted from: A. Sahu and J. D. Lambris Immunopharmacology 49 (2000) 133-148.

The invention thus relates in particular to the use of the (poly)peptides having SCIN activity for the manufacture of a preparation for diagnosis, prophylaxis or therapy, in particular for the treatment of acute and chronic inflammation reactions, more in particular for the treatment of the indications referred to above.

Also part of the present invention is the use of therapeutic compositions comprising a suitable excipient and one or more of the (poly)peptides having SCIN activity.

According to the invention the SCIN-polypeptide is used for the preparation of a medicament for use in a method for treating a subject suffering from inflammation by administering a therapeutically effective amount of the said SCIN-(poly)peptide, as well as in a method for treating a subject suffering from staphylococcus infection by administering a therapeutically effective amount of an antibody and/or biologically active fragment thereof.

According to a further aspect thereof, the invention relates to the use of micro-organisms expressing one or more nucleic acid molecules encoding a SCIN-(poly)peptide for the preparation of a medicament for the treatment of acute and chronic inflammation reactions, such as listed in Table 2.

As an alternative to SCIN the present invention can be practised with peptoids and peptidomimetics designed on the basis of SCIN (poly)peptides.

Various definitions for peptidomimetics have been formulated in literature. Among others, peptidomimetics have been described as “chemical structures designed to convert the information contained in peptides into small non-peptide structures”, “molecules that mimic the biological activity of peptides but no longer contain any peptide bonds”, “structures which serve as appropriate substitutes for peptides in interactions with receptors and enzymes” and as “chemical Trojan horses”.

In general, peptidomimetics can be classified into two categories. The first consists of compounds with non-peptide-like structures, often scaffolds onto which pharmacophoric groups have been attached. Thus, they are low molecular-weight compounds and bear no structural resemblance to the native peptides, resulting in an increased stability towards proteolytic enzymes.

The second main class of peptidomimetics consists of compounds of a modular construction comparable to that of (poly)peptides. These compounds can be obtained by modification of either the (poly)peptide side chains or the (poly)peptide backbone. Peptidomimetics of the latter category can be considered to be derived of (poly)peptides by replacement of the amide bond with other moieties. As a result, the compounds are expected to be less sensitive to degradation by proteases. Modification of the amide bond also influences other characteristics such as lipophilicity, hydrogen bonding capacity and conformational flexibility, which in favourable cases may result in an overall improved pharmacological and/or pharmaceutical profile of the compound.

Oligomeric peptidomimetics can in principle be prepared starting from monomeric building blocks in repeating cycles of reaction steps. Therefore, these compounds may be suitable for automated synthesis analogous to the well-established preparation of peptides in peptide synthesizers. Another application of the monomeric building blocks lies in the preparation of peptide/peptidomimetic hybrids, combining natural amino acids and peptidomimetic building blocks to give products in which only some of the amide bonds have been replaced. This may result in compounds which differ sufficiently from the native peptide to obtain an increased biostability, but still possess enough resemblance to the original structure to retain the biological activity.

Suitable peptidomimetic building blocks for use in the invention are amide bond surrogates, such as the oligo-β-peptides (Juaristi, E. Enantioselective Synthesis of b-Amino Acids; Wiley-VCH: New York, 1996), vinylogous peptides (Hagihari, M. et al., J. Am. Chem. Soc. 1992, 114, 10672-10674), peptoids (Simon, R. J. et al., Proc. Natl. Acad. Sci. USA 1992, 89, 9367-9371; Zuckermann, R. N. et al., J. Med. Chem. 1994, 37, 2678-2685; Kruijtzer, J. A. W. & Liskamp, R. M. J. Tetrahedron Lett. 1995, 36, 6969-6972); Kruijtzer, J. A. W. Thesis; Utrecht University, 1996; Kruijtzer, J. A. W. et al., Chem. Eur. J. 1998, 4, 1570-1580), oligosulfones (Sommerfield, T. & Seebach, D. Angew. Chem., Int. Ed. Eng. 1995, 34, 553-554), phosphodiesters (Lin, P. S.; Ganesan, A. Bioorg. Med. Chem. Lett. 1998, 8, 511-514), oligosulfonamides (Moree, W. J. et al., Tetrahedron Lett. 1991, 32, 409-412; Moree, W. J. et al., Tetrahedron Lett. 1992, 33, 6389-6392; Moree, W. J. et al., Tetrahedron 1993, 49, 1133-1150; Moree, W. J. Thesis; Leiden University, 1994; Moree, W. J. et al., J. Org. Chem. 1995, 60, 5157-5169; de Bont, D. B. A. et al., Bioorg. Med. Chem. Lett. 1996, 6, 3035-3040; de Bont, D. B. A. et al., Bioorg. Med. Chem. 1996, 4, 667-672; Löwik, D. W. P. M. Thesis; Utrecht University, 1998), peptoid sulfonamides (van Ameijde, J. & Liskamp, R. M. J. Tetrahedron Lett. 2000, 41, 1103-1106), vinylogous sulfonamides (Gennari, C. et al., Eur. J. Org. Chem. 1998, 2437-2449), azatides (or hydrazinopeptides) (Han, H. & Janda, K. D. J. Am. Chem. Soc. 1996, 118, 2539-2544), oligocarbamates (Paikoff, S. J. et al., Tetrahedron Lett. 1996, 37, 5653-5656; Cho, C. Y. et al., Science 1993, 261, 1303-1305), ureapeptoids (Kruijtzer, J. A. W. et al., Tetrahedron Lett. 1997, 38, 5335-5338; Wilson, M. E. & Nowick, J. S. Tetrahedron Lett. 1998, 39, 6613-6616) and oligopyrrolinones (Smith III, A. B. et al., J. Am. Chem. Soc. 1992, 114, 10672-10674). FIG. 22 shows the structures of these peptidomimetic building blocks.

The vinylogous peptides and oligopyrrolinones have been developed in order to be able to form secondary structures (β-strand conformations) similar to those of peptides, or mimic secondary structures of peptides. All these oligomeric peptidomimetics are expected to be resistant to proteases and can be assembled in high-yielding coupling reactions from optically active monomers (except the peptoids).

Peptidosulfonamides are composed of α- or β-substituted amino ethane sulfonamides containing one or more sulfonamide transition-state isosteres, as an analog of the hydrolysis of the amide bond. Peptide analogs containing a transition-state analog of the hydrolysis of the amide bond have found a widespread use in the development of protease inhibitor e.g. HIV-protease inhibitors.

Another approach to develop oligomeric peptidomimetics is to completely modify the peptide backbone by replacement of all amide bonds by non-hydrolyzable surrogates e.g. carbamate, sulfone, urea and sulfonamide groups. Such oligomeric peptidomimetics may have an increased metabolic stability. Recently, an amide-based alternative oligomeric peptidomimetics has been designed viz. N-substituted Glycine-oligopeptides, the so-called peptoids. Peptoids are characterized by the presence of the amino acid side chain on the amide nitrogen as opposed to being present on the α-C-atom in a peptide, which leads to an increased metabolic stability, as well as removal of the backbone chirality. The absence of the chiral α-C atom can be considered as an advantage because spatial restrictions which are present in peptides do not exist when dealing with peptoids. Furthermore, the space between the side chain and the carbonyl group in a peptoid is identical to that in a peptide. Despite the differences between peptides and peptoids, they have been shown to give rise to biologically active compounds.

Translation of a (poly)peptide chain into a peptoid peptidomimetic may result in either a peptoid (direct-translation) or a retropeptoid (retro-sequence). In the latter category the relative orientation of the carbonyl groups to the side chains is maintained leading to a better resemblance to the parent peptide.

Review articles about peptidomimetics that are incorporated herein by reference are:

Adang, A. E. P. et al.; Recl. Trav. Chim. Pays-Bas 1994, 113, 63-78; Giannis, A. & Kolter, T. Angew. Chem. Int. Ed. Engl. 1993, 32, 1244-1267; Moos, W. H. et al., Annu. Rep. Med. Chem. 1993, 28, 315-324; Gallop, M. A. et al., J. Med. Chem. 1994, 37, 1233-1251; Olson, G. L. et al., J. Med. Chem. 1993, 36, 3039-30304; Liskamp, R. M. J. Recl. Trav. Chim. Pays-Bas 1994, 113, 1-19; Liskamp, R. M. J. Angew. Chem. Int. Ed. Engl. 1994, 33, 305-307; Gante, J. Angew. Chem. Int. Ed. Engl. 1994, 33, 1699-1720; Gordon, E. M. et al., Med. Chem. 1994, 37, 1385-1401; and Liskamp, R. M. J. Angew. Chem. Int. Ed. Engl. 1994, 33, 633-636.

The invention thus furthermore relates to the use of molecules that are not SCIN (poly)peptides themselves but have a structure and function similar to those of the SCIN (poly)peptides described herein. Examples of such molecules are the above described peptidomimetics, but also compounds in which one or more of the amino acids are replaced by non-proteinogenic amino acids or D-amino acids. When reference is made in this application to (poly)peptides, it is intended to include also such other compounds that have a similar or the same structure and function and as a consequence a similar or the same biological SCIN activity as the (poly)peptides.

More in particular substitutions can be made with non-proteinogenic amino acids selected from the group consisting of 2-naphtylalanine (Nal(2)), β-cyclohexylalanine (Cha), p-amino-phenylalanine ((Phe(p-NH₂), p-benzoyl-phenylalanine (Bpa), ornithine (Orn), norleucine (Nle), 4-fluoro-phenylalanine (Phe(p-F)), 4-chloro-phenylalanine (Phe(p-Cl)), 4-bromo-phenylalanine (Phe(p-Br)), 4-iodo-phenylalanine (Phe(p-I)), 4-methyl-phenylalanine (Phe(p-Me)), 4-methoxy-phenylalanine (Tyr(Me)), 4-nitro-phenylalanine (Phe(p-NO2)).

Suitable D-amino acids for substituting the amino acids in the (poly)peptides of the invention are for example those that are selected from the group consisting of D-phenylalanine, D-alanine, D-arginine, D-asparagine, D-aspartic acid, D-cysteine, D-glutamic acid, D-glutamine, D-histidine, D-isoleucine, D-leucine, D-lysine, D-methionine, D-proline, D-serine, D-threonine, D-tryptophan, D-tyrosine, D-valine, D-2-naphtylalanine (D-Nal(2)), β-cyclohexyl-D-alanine (D-Cha), 4-amino-D-phenylalanine (D-Phe(p-NH₂)), p-benzoyl-D-phenylalanine (D-Bpa), D-Ornithine (D-Orn), D-Norleucine (D-Nle), 4-fluoro-D-phenylalanine (D-Phe(p-F)), 4-chloro-D-phenylalanine (D-Phe(p-Cl)), 4-bromo-D-phenylalanine (D-Phe(p-Br)), 4-iodo-D-phenylalanine (D-Phe(p-I)), 4-methyl-D-phenylalanine (D-Phe(p-Me)), 4-methoxy-D-phenylalanine (D-Tyr(Me)), 4-nitro-D-phenylalanine (D-Phe(p-NO2)).

One or more of the amino acids in the (poly)peptides can be replaced by peptoid building blocks, e.g. selected from the group consisting of N-substituted glycines, such as N-benzylglycine (NPhe), N-methylglycine (NAla), N-(3-guanidinopropyl)glycine (NArg), N-(Carboxymethyl)glycine (NAsp), N-(carbamylmethyl)glycine (NAsn), N-(thioethyl)-glycine (NhCys), N-(2-carboxyethyl)glycine (NGlu), N-(2-carbamylethyl)glycine (NGln), N-(imidazolylethyl)glycine (NhHis), N-(1-methylpropyl)glycine (NIle), N-(2-methylpropyl)glycine (NLeu), N-(4-aminobutyl)glycine (NLys), N-(2-methylthioethyl)glycine (NMet), N-(hydroxyethyl)glycine (NhSer), N-(2-hydroxypropyl)glycine (NhThr), N-(3-indolylmethyl)glycine (NTrp), N-(p-hydroxyphenmethyl)-glycine (NTyr), N-(1-methylethyl)glycine (NVal).

All compounds for use according to the invention may also be in cyclic form. A cyclic compound may have improved potency, stability, rigidity and/or other pharmaceutical and/or pharmacological characteristics.

All molecules that are used according to the invention can be labelled in any way. Examples of labelling include but are not limited to fluorescence, biotin, radioactive labelling etc. Such labelled molecules can be used for tracing SCIN protein in an organism.

The present invention will be further illustrated in the examples that follow and that are in no way intended to be limiting to this invention. In this description and the examples reference is made to the following figures:

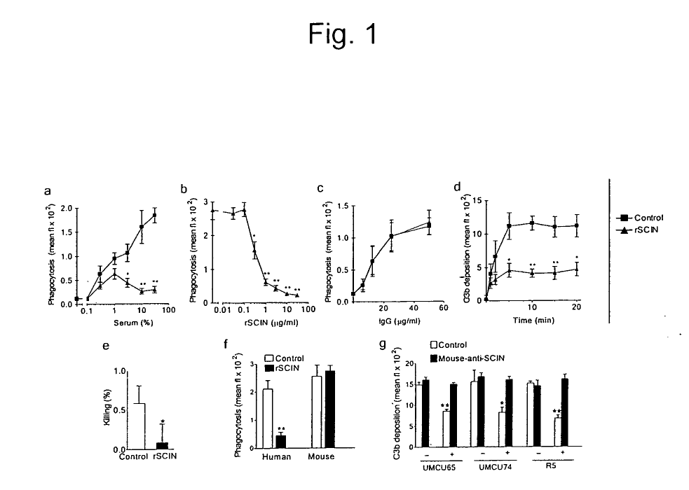

FIG. 1. SCIN inhibits phagocytosis and killing of S. aureus by interfering with human complement. (a-c), The effect of rSCIN on phagocytosis of S. aureus by human neutrophils (a) at different concentrations of human serum with 3 μg/ml rSCIN, (b) at 10% human serum with different rSCIN concentrations or (c) with purified human IgG and 10 μg/ml rSCIN. (d) C3b deposition on S. aureus at 10% human serum and 3 μg/ml rSCIN. (e) Neutrophil bacterial killing at 40% human serum and 10 μg/ml rSCIN. (f) Phagocytosis of S. aureus by human neutrophils in 30% mouse or human serum with 10 μg/ml rSCIN. (g) C3b deposition on S. aureus in 10% serum in the presence of supernatants of SaPI5-positive (+) and isogenic SaPI5-negative S. aureus (−). Supernatants were pre-incubated with mouse anti-SCIN antibodies. Mean fl=mean fluorescence. All data shown represent mean±SE of three separate experiments. *, P<0.05 versus control; **, P<0.005 versus control.

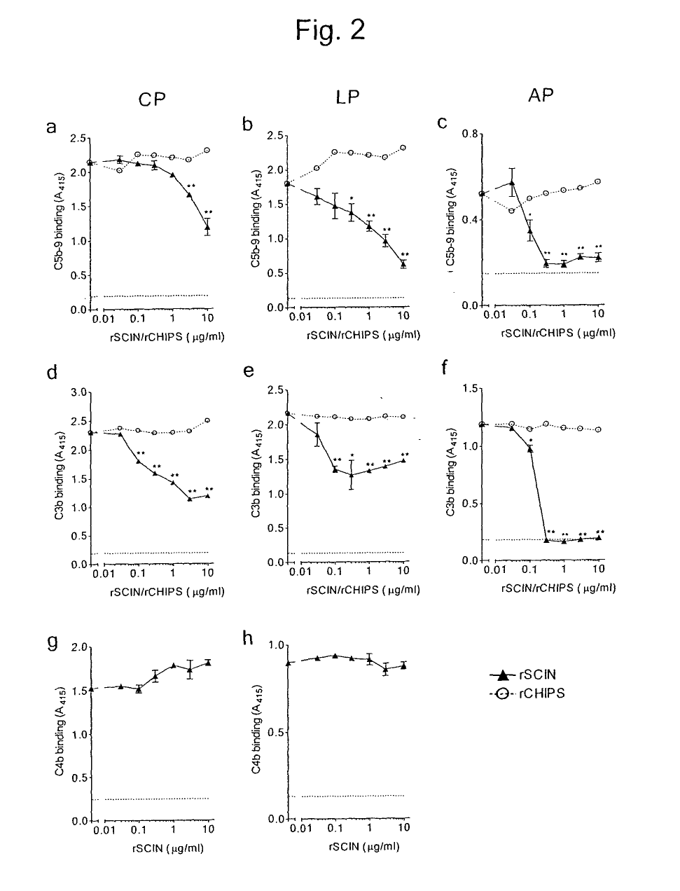

FIG. 2. SCIN inhibits all complement pathways. (a-c) C5b-9 deposition via the CP (a, 1% serum), LP (b, 1% serum) and AP (c, 20% serum). (d-f) C3b deposition via the CP (d, 2% serum), LP (e, 1% serum) and AP (f, 20% serum). To exclude involvement of the AP in the CP and LP, factor D-deficient serum was tested, showing 50% inhibition by rSCIN (data not shown). (g-h) CP and LP-mediated C4b deposition (0.5% and 2% serum respectively). Anti-C1q and anti-MBL antibodies did inhibit C4b deposition in the CP and LP (data not shown). Straight dotted lines present the background signal in the absence of serum. All data are mean±SD from one representative out of three separate experiments. *, P<0.05 versus control; **, P<0.005 versus control.

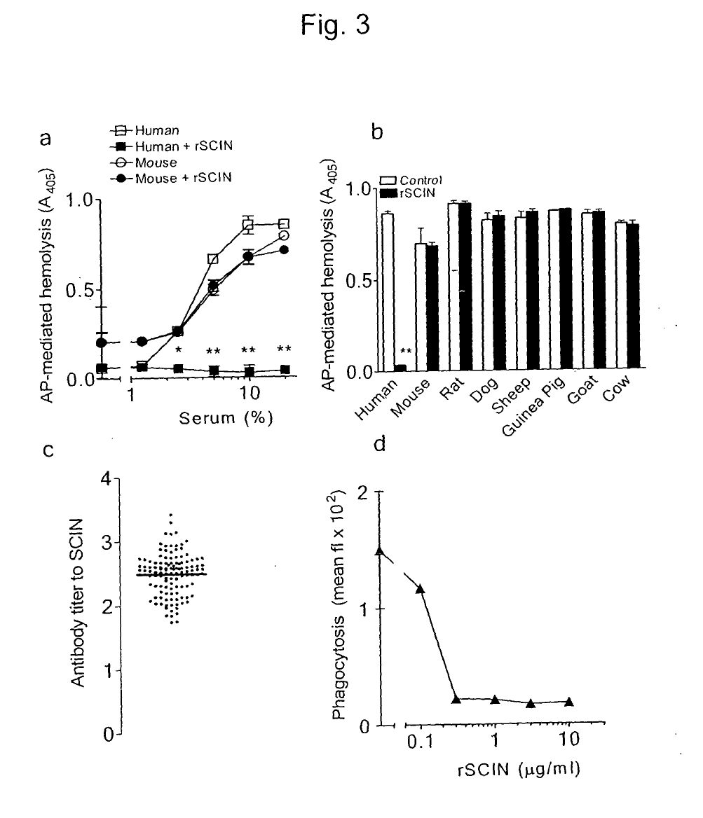

FIG. 3. SCIN is a human-specific complement inhibitor. (a-b) Alternative pathway mediated hemolysis of rabbit erythrocytes in (a) various concentration of mouse and human serum or in (b) 20% mouse, rat, dog, sheep, guinea pig, goat and cow serum with rSCIN at 10 μg/ml. Data represent mean±SE of three separate experiments. *, P<0.05 versus control; **, P<0.005 versus control. (c) Antibody titer to SCIN in sera of healthy individuals and patients. (d) Phagocytosis of S. aureus in human whole blood. For c and d, representatives of three separate experiments are depicted.

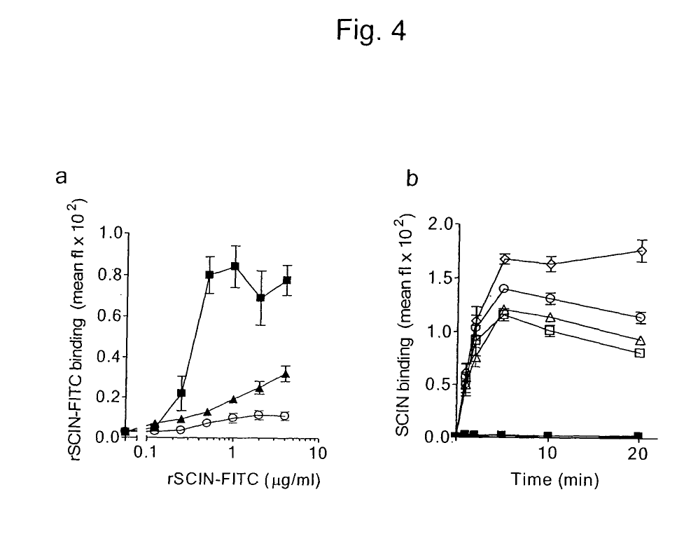

FIG. 4. SCIN binds to particles in a serum- and temperature dependent way. (a) Binding of rSCIN-FITC to zymosan in 10% serum at 37° C. (▪). No binding in the absence of serum at 37° C. (▴) or in the presence of serum at 0° C. (◯). (b) SCIN from staphylococcal supernatants binds to S. aureus during opsonization in 10% serum. SCIN was detected with murine anti-SCIN antibodies. Binding was only observed with supernatants of SaPI5-positive strains, but not with supernatants of their isogenic SaPI5-negative variants. Explanation of symbols: rSCIN at 2 μg/ml (⋄), UMCU65 (▴), UMCU65+SaPI5 (Δ), UMCU74 (), UMCU74+SaPI5 (◯), R5 (▪), R5+SaPI5 (□). Data shown represent mean±SE of three separate experiments.

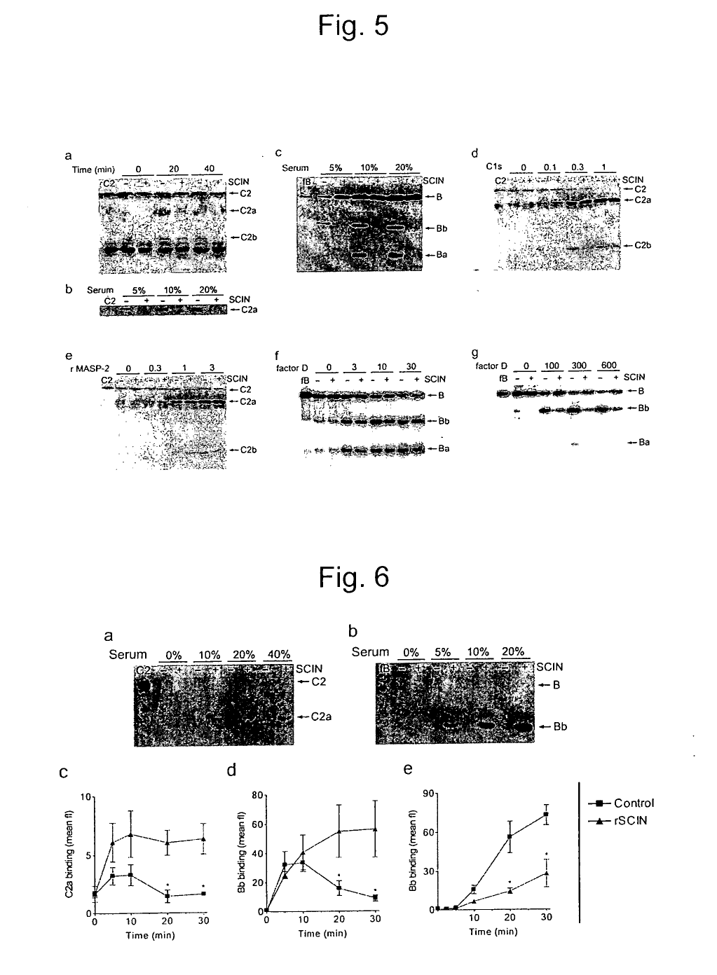

FIG. 5. SCIN interferes with formation of C3 convertases. (a-b) Detection of C2-cleavage products in serum after incubation with zymosan, rSCIN at 10 μg/ml. In b, a different batch of polyclonal anti-C2 antibody was used for better visualization of C2a in controls. (c) Detection of fB-cleavage products in serum after incubation with S. aureus. (d-e) C1s- or rMASP-2-mediated cleavage of C2 in the absence of an activator surface, rSCIN at 10 μg/ml. C1s (d) in μg/ml, rMASP-2 (e) in ng/ml. (f) fD-mediated cleavage of fB in the presence of C3(H₂O), rSCIN at 10 μg/ml. Factor D in ng/ml. (g) fD-mediated cleavage of fB in the presence of pre-opsonized zymosan, rSCIN at 3 μg/ml. Factor D in ng/ml. All blots are representatives of three separate experiments.

FIG. 6. SCIN prevents dissociation of C3 convertases. (a) Surface detection of C2 split products on zymosan after opsonization in serum, rSCIN at 10 μg/ml). (b) Surface detection of fB split products on S. aureus after opsonization in serum, rSCIN at 10 μg/ml). (c-d) Surface detection of C2a (c) or Bb (d) on zymosan after incubation with 20% serum, rSCIN at 10 μg/ml. (e) Surface detection of Bb on zymosan after incubation with 20% serum in the presence of properdin and Ni²⁺ to stabilize C3bBb in controls, rSCIN at 10 μg/ml). Blots were prepared using polyclonal antibodies against C2 and fB and are representatives of three separate experiments. For flow cytometry, monoclonal antibodies against C2a and Bb were used. Data shown in c-e represent mean±SE of three separate experiments. *, P<0.05 versus control.

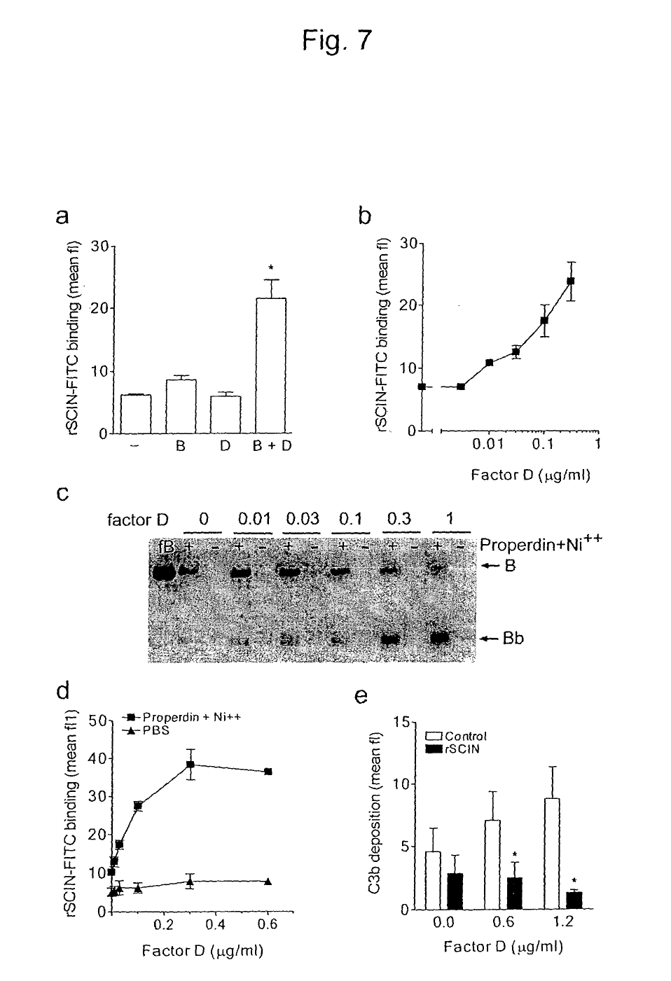

FIG. 7. SCIN binds and inactivates C3bBb. Binding of rSCIN-FITC (3 μg/ml) to pre-opsonized zymosan in the presence of buffer(−), fB (B, 40 μg/ml), fD (D, 0.6 μg/ml) or both (B+D). (b) Binding of rSCIN-FITC (3 μg/ml) to pre-opsonized zymosan in the presence of fB (40 μg/ml) and increasing concentrations of fD. (c) Stabilization of C3bB and C3bBb on zymosan in the presence of properdin (4 μg/ml) and 2 mM NiCl₂ (+; P+Ni⁺⁺) compared to PBS (−). (d) Binding of rSCIN-FITC (3 μg/ml) to C3bBbP on zymosan. (e) C3b deposition on zymosan by C3bBbP, rSCIN at 3 μg/ml. Graphs represent mean SE of three separate experiments. *, P<0.05 versus control. Blots are representatives of three separate experiments.

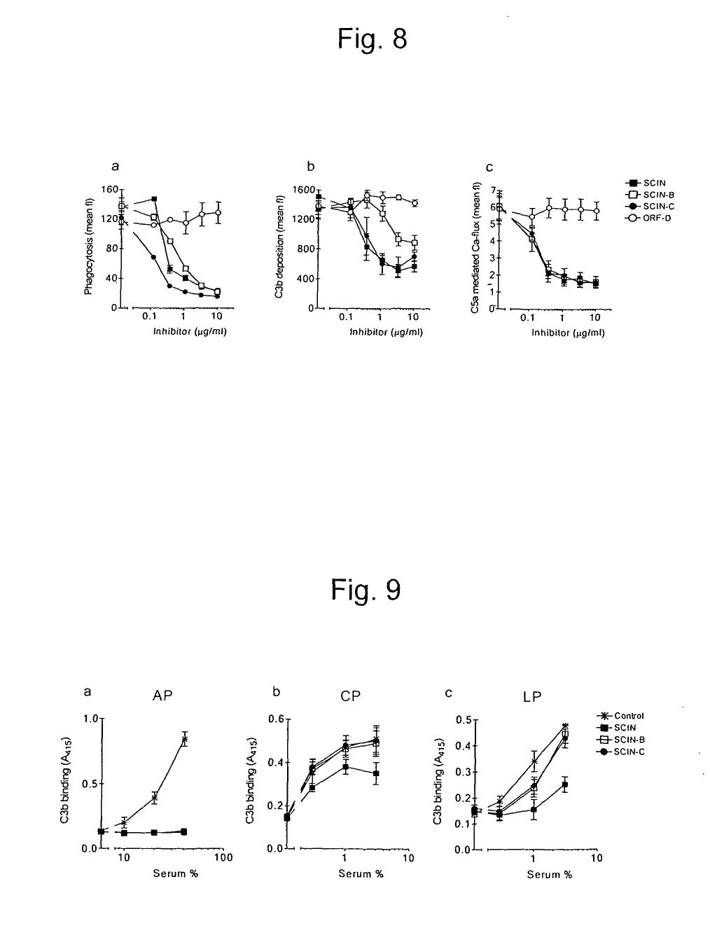

FIG. 8. SCIN-B and SCIN-C inhibit phagocytosis and complement activation. (a) The effect of SCIN homologues on phagocytosis of S. aureus by human neutrophils in 10% human serum. (b) C3b deposition on S. aureus at 10% human serum and different concentrations of SCIN homologues. (c) C5a-mediated calcium mobilization by bacterial supernatants after opsonization in 10% human sera in the presence of SCIN homologues. Mean fl=mean fluorescence. All data shown represent mean±SE of three separate experiments. *, P<0.05 versus control; **, P<0.005 versus control.

FIG. 9. SCIN-B and SCIN-C efficiently prevent alternative pathway activation. The effect of SCIN, SCIN-B and SCIN-C (all at 10 μg/ml) on C3b deposition via the (a) CP, (b) LP or (c) AP measured by ELISA. All data shown represent mean±SE of three separate experiments. *, P<0.05 versus control; **, P<0.005 versus control.

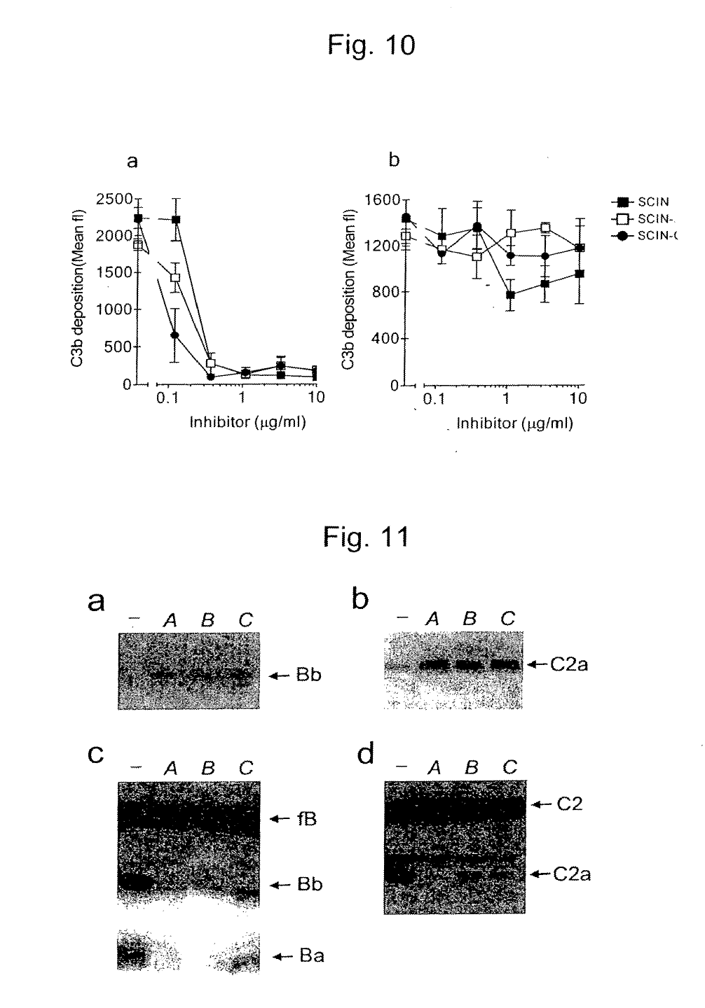

FIG. 10. SCIN-B and SCIN-C prevent C3b deposition via the alternative pathway. The effect of SCIN, SCIN-B and SCIN-C on C3b deposition on S. aureus. (a)-AP-mediated C3b deposition in 10% human sera in the presence of Mg-EGTA (b) CP-LP-mediated C3b deposition in 10% factor D-deficient sera. All data shown represent mean±SE of three separate experiments. *, P<0.05 versus control; **, P<0.005 versus control.

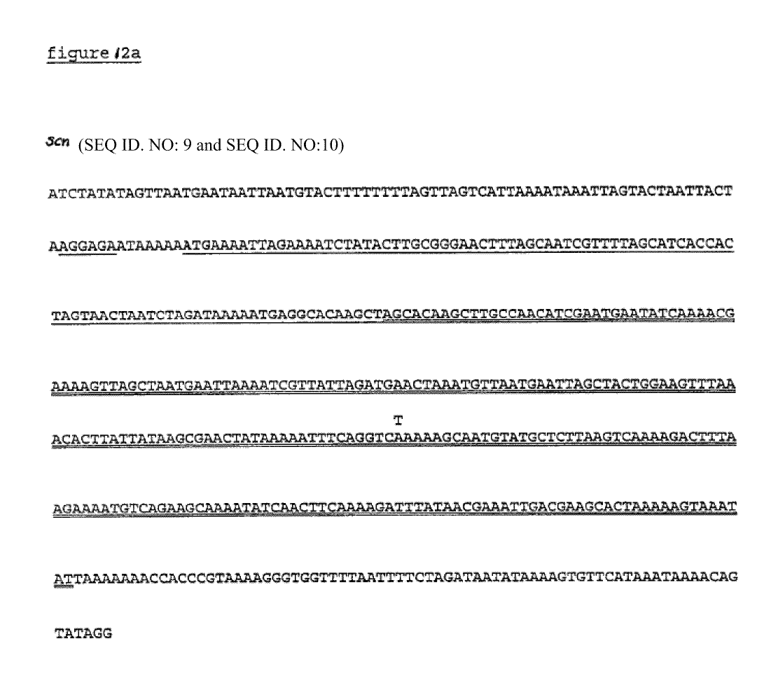

FIG. 11. SCIN-B and SCIN-C act on C3 convertases. (a-b) Stabilization of C3 convertases on S. aureus in the presence of buffer (−), SCIN (A), SCIN-B (B) and SCIN-C (C), detected by immunoblotting. (a) Detection of Bb on staphylococci after opsonization in 20% human sera for 30 minutes. (b) Detection of C2a on staphylococci after opsonization in 20% human sera for 20 minutes. (c-d) Formation of C3 convertases on S. aureus. Analysis of fB and C2 cleavage products in supernatants after opsonization in the presence of buffer (−), SCIN (A), SCIN-B (B) and SCIN-C (C) by immunoblotting. (c) fB cleavage products in supernatants after opsonization of staphylococci in 5% human sera for 30 minutes. (d) C2 cleavage products in supernatants after opsonization of staphylococci in 20% human sera for 20 minutes.

FIG. 12 shows the sequence of SCIN, SCIN-B and SCIN-C genes.

FIG. 13 shows the amino acids sequence deduced for the SCIN, SCIN-B and SCIN-C genes.

### EXAMPLES

### Example 1

### Staphylococcal Complement Inhibitor Acting on C3 Convertase

### Introduction

The complement system plays a pivotal role in host defense but also contributes to tissue injury in several diseases. The assembly of C3 convertases (C4b2a and C3bBb) is a prerequisite for complement activation. The convertases catalyze C3b deposition on activator surfaces.

This example describes that Staphylococcal Complement Inhibitor (SCIN), an excreted 9.8 kD protein, blocks human complement by specific interaction with C4b2a and C3bBb. SCIN binds and stabilizes C3 convertases, interfering with C3b deposition via the classical, lectin and alternative complement pathway. This leads to a dramatic decrease in phagocytosis and killing of Staphylococcus aureus by human neutrophils. As a highly active and small soluble protein that acts exclusively on surfaces, SCIN is a promising anti-inflammatory molecule.

### Methods

### SCIN

Cloning and expression of scn was carried out as described for CHIPS mutants (Haas et al., J. Immunol. 173:5704-5711 (2004)) with use of the following primers: 5′-GAGCACAAGCTTGCCAACATCG-3′ (forward) and 5′GGAATTCCTTAATATTTACTTTTT-3′ (reversed, containing EcoRI site (underlined)). Recombinant SCIN (rSCIN) and CHIPS (rCHIPS) were purified as described (de Haas et al., J. Exp. Med. 199: 687-695 (2004)).

### Anti-SCIN Antibodies

Mouse-anti-SCIN and rabbit-anti-SCIN antibodies were prepared as described earlier (Haas et al., supra). Four different mouse-anti-SCIN monoclonal antibodies were used in this study: three blocking antibodies (2F4, 2B12, 3F1) completely inhibit SCIN activity in all complement pathways, they do not recognize SCIN bound to an opsonized particle. One antibody (3G3) did recognize SCIN when-bound to an opsonized particle, partially blocks activity, and was used for detection of SCIN at the bacterial surface.

S. aureus Strains

Laboratory S. aureus strains Cowan, Cowan EMS, COL, Wood 46 and R5 were used, next to 2 blood isolates (UMCU65 and UMCU74) obtained within the UMC Utrecht. S. aureus SaPI5-negative strains R5, UMCU65 and UMCU74 were infected with SaPI5-carrying bacteriophage Phi13 as described (van Wamel et al., J. Bacteriol 188: 1310-1315 (2006)). PCR and southern blotting revealed the successful integration of SaPI5 type E (scn-sak) in R5 and UMCU74, and of SaPI5 type B (scn-chp-sak) in UMCU65. Culture supernatants of UMCU65+SaPI5, UMCU74+SaPI5 and R5+SaPI5 contained 2.3 μg/ml, 1.7 μg/ml and 3.5 μg/ml of SCIN respectively as determined by ELISA (de Haas et al., supra).

Phagocytosis, Killing and C3b Deposition on S. aureus

Informed consent was obtained from all subjects. The study protocol was approved by the medical ethics committee of the University Medical Center Utrecht. Phagocytosis experiments were performed as described (Rooijakkers et al., Microbes Infect. 7, 476-484 (2005)) incubating 2.5×10⁵ human neutrophils and FITC-labeled S. aureus strain Cowan EMS, Cowan, COL, Wood 46, UMCU65 or UMCU74 (2.5×10⁶) with human serum or IgG, and rSCIN for 15 min at 37° C. Phagocytosis in human whole blood was performed as described (Mollnes et al., Blood 100:1869-1877 (2002)). For killing, S. aureus strain Cowan EMS (5×10⁴) was pre-opsonized with 40% human serum for 15 min and subsequently incubated with 1×10⁶ neutrophils for 1 h at 37° C. Killing was analyzed on Luria agar: [(CFUt60-CFUt0): CFUt0]×100. For C3b deposition, 3×10⁶ S. aureus Cowan EMS were incubated with serum and rSCIN or bacterial supernatants in 20 mM HEPES, 140 mM NaCl, 5 mM CaCl₂, 2.5 mM MgCl₂, 0.1% (v/v) Tween-20, pH 7.4 (Hepes-Buffered Saline with Ca²⁺ and Mg²⁺ (HBS++)) at 37° C. Supernatants were pre-incubated with 65 μg/ml anti-SCIN monoclonal antibody (2B12). Surface-bound C3b/iC3b were detected using FITC-conjugated goat F(ab′)2 anti-human-C3 antibodies (Protos Immunoresearch, San Francisco, Ala.). Antibody binding to 10.000 bacteria was measured by flow cytometry.

### Complement Assays

Functional activity of the CP, LP and AP was screened as described previously (Rooijakkers et al., Nat. Immunol. 6:920-927 (2005)). Sera were pre-incubated with rSCIN for 15 min at room temperature. Alternative pathway hemolytic assay was performed by incubation of 2×10⁷ rabbit erythrocytes (Biotrading, Wilnis, The Netherlands) with serum in Veronal buffered Saline (VBS) containing 5 mM MgCl₂ and 10 mM EGTA for 1 h at 37° C. Cells were spun down and A405 measurements of supernatants were taken.

### Distribution of Anti-SCIN Antibodies

Distribution of anti-SCIN antibodies in human sera was determined as described earlier for anti-SAK antibodies (Rooijakkers et al., Microbes and Infection 7:476-484 (2005)). Sera were obtained from 80 blood bank donors, lab donors, and 20 patients suffering from recurrent staphylococcal infections (treated at the UMC Utrecht) after informed consent.

### Analysis of C2 and fB

Zymosan (5 μg, Sigma) or 5×10⁶ S. aureus Cowan EMS were incubated with serum for 20 min at 37° C. in HBS⁺⁺ or in HBS− 2 mM MgCl₂-2 mM EGTA respectively. Then, after centrifugation both the supernate and particle-associated proteins were subjected to SDS-PAGE and analyzed by immunoblotting (Rooijakkers et al., supra). Human C2 and fB were detected with goat anti-human C2 (Quidel Corporation, San Diego, Calif.) or goat-anti human factor B (Merck, Darmstadt, Germany), followed by peroxidase-conjugated anti-goat IgG (Santa Cruz Biotechnology, Santa Cruz, Calif.).

Alternatively, 250 μg zymosan was incubated with 10% serum at 37° C. in VBS containing Ca²⁺ and Mg² (VBS⁺⁺)-0.05% BSA and surface-bound C2a and Bb were detected using mouse anti-C2a (Antibodyshop, Gentofte, Denmark) or mouse anti-Bb (Quidel) antibodies and FITC-labeled goat anti-mouse antibodies (Dako, Glostrup, Denmark).

For stabilization of C3bBb, zymosan was mixed with 5% serum in the presence of 2 mM NiCl₂ and 10 μg/ml properdin (Merck). Human C2 (50 ng, Merck) was incubated with human C1s (He et al., FEBS Letters 412:506-510 (1997)) or recombinant human MASP-2 (consisting of CCP1-CCP2-SP domains (Ambrus et al., J. Immunol. 170:1374-1382 (2003)) in HBS⁺⁺ for 30 min at 37° C. For fluid-phase cleavage of fB, C3 (250 μg/ml), fB (40 μg/ml) (both purified as described in Schreiber et al., PNAS 75:3948-3952 (1978)) and fD (0-1 μg/ml, Merck) were mixed in PBS-MgCl₂ for 30 min at 37° C. To prepare pre-opsonized zymosan, 250 μg zymosan was incubated with 20% serum in PBS for 30 min at 37° C. After washing, zymosan was incubated with fB (40 μg/ml), fD (0-0.6 μg/ml) and rSCIN for 30 min at 37° C.

### Binding of SCIN to Particles

Zymosan (250 μg) or 3×10⁷ S. aureus Cowan EMS was incubated with FITC-labeled SCIN (de Haas et al., supra)) or bacterial supernatants and 10% serum for 20 min in VBS containing Ca²⁺ and Mg² (VBS⁺⁺)-0.05% BSA. Alternatively, SCIN was detected with mouse-anti-SCIN (3G3) and FITC-labeled goat anti-mouse antibodies. Pre-opsonized zymosan was incubated with rSCIN-FITC (3 μg/ml) and purified fB (40 μg/ml) and fD (0-0.6 μg/ml). Zym-C3bBbP was prepared by incubation of zym-C3b with fB (40 μg/ml), fD (0-0.6 μg/ml) and properdin (4 μg/ml) in PBS-2 mM NiCl₂-25 mM NaCl-1% BSA (Therman et al., Mol. Immunol. 42: 87-97 (2005)) or in the absence of properdin with PBS-1% BSA. Zym-C3bBbP was washed with PBS-2 mM NiCl₂-25 mM NaCl-1% BSA and incubated with rSCIN-FITC (3 μg/ml) in the same buffer with 4 μg/ml properdin for 30 min at 37° C. Alternatively, Zym-C3bBbP was incubated with non-labeled rSCIN (3 μg/ml) for 5 min at 37° C. followed by 25 min incubation with FITC-labeled C3 (20 μg/ml) at 37° C. Finally, particles were washed and mean fluorescence of 10,000 particles was determined by flow cytometry.

### Statistical Analysis

Statistics were performed using the Student's T-test; P values of <0.05 were regarded as significant.

### Accession Numbers

The accession numbers for SCIN are gi|14247715|dbj|BAB58104.1| and gi|13701735|dbj|BAB43028.1|. Accession numbers for SCIN homologues in S. aureus are: SCIN-like B: gi|13700958|dbj|BAB42254.1|, SCIN-like C: gnl|Sanger 159288|Staphylococcus (from the Sanger database (S. aureus 252, (MRSA 16) and SCIN-like D: gi|21203369|dbj|BAB94070.1|.).

### Results

### Identification of SCIN

The gene for SCIN (scn) is located on the bacteriophage-located SaPI5 and was found in 90% of S. aureus strains. Due to its unique location on an immune evasion cluster, recombinant SCIN (rSCIN) was prepared to study its role in immune evasion. The SCIN gene consists of 348 base pairs that encode a protein of 116 amino acids. Following cleavage of a typical secretion signal-peptide, the excreted protein has a molecular mass of 9.8 kD.

### SCIN Inhibits Phagocytosis and C3b Deposition

Phagocytes recognize foreign particles most efficiently after opsonization with serum-derived opsonins such as IgG and C3b or inactive C3b (iC3b). In an in vitro phagocytosis assay, it was observed that rSCIN efficiently blocks bacterial uptake by human neutrophils. Neutrophils were incubated with S. aureus in the presence of human serum and rSCIN (FIGS. 1a,b). Confocal microscopy confirmed that bacteria were inside neutrophils. Recombinant SCIN (3 μg/ml) strongly inhibited phagocytosis at serum concentrations of 3% and higher (FIG. 1a).

In 10% serum, rSCIN reduced phagocytosis dose-dependently with 50% inhibition at 0.3 μg/ml (FIG. 1b). This inhibition was observed for all tested staphylococcal strains. Pre-incubation of neutrophils with rSCIN did not alter phagocytosis indicating that SCIN did not interact with neutrophils but affected opsonization.

Furthermore, it was observed that rSCIN (10 μg/ml) did not influence phagocytosis in 40% complement-inactivated serum and also failed to interfere with IgG-mediated phagocytosis (FIG. 1c), indicating that SCIN prevents complement-mediated phagocytosis. Using anti-C3 antibodies it was shown that rSCIN (3 μg/ml) indeed prevented C3b deposition on S. aureus (FIG. 1d). Moreover it was shown that rSCIN (10 μg/ml) reduced neutrophil bacterial killing (FIG. 1e). In the presence of mouse serum, rSCIN did not inhibit phagocytosis suggesting that SCIN is human specific, like the other molecules on SaPI5 (FIG. 1f). When bacterial opsonization was performed in the presence of supernatants of SaPI5-positive S. aureus strains a reduced amount of C3b molecules on the bacterial surface was observed when compared to supernatants of isogenic SaPI5-negative S. aureus strains. Using neutralizing anti-SCIN antibodies, it was demonstrated that the excretion of SCIN by S. aureus led to the efficient reduction of bacterial opsonization (FIG. 1g).

Taken together these data demonstrate that SCIN efficiently inhibits phagocytosis, killing and opsonization of S. aureus in human serum. Therefore, staphylococci can prevent opsonization and complement-mediated phagocytosis by excreting SCIN.

### SCIN Inhibits all Complement Pathways

All complement pathways are involved in the host defense against S. aureus. To evaluate the effects of SCIN on each pathway separately, SCIN was tested in an ELISA-based system in which each pathway is analyzed without cross-interaction (Roos et al., Mol. Immunol. 39: 655-668 (2003))). The assays are based on specific coatings for each pathway in combination with specific buffer systems (Seelin et al., J. Immunol. Meth. 296:187-198 (2005)). Complement activation was evaluated by detection of deposited C4b, C3b and C5b-9. Recombinant SCIN interfered with activation of all three pathways at the level of C5b-9 and C3b deposition (FIG. 2a-f). In the CP and LP, rSCIN inhibited C5b-9 and C3b deposition by 50% at 10 μg/ml (FIG. 2a-b,d-e). No inhibition of C4b deposition was found by the CP and LP (FIGS. 2g,h). In the AP, C5b-9 and C3b deposition were almost completely blocked at 0.3 μg/ml of rSCIN and higher (FIGS. 2c,f). All pathways were also inhibited at other tested serum concentrations, using rSCIN at 10 μg/ml (data not shown).

In summary, SCIN prevents proper activation of all complement pathways. C3b deposition via the classical and lectin pathway is inhibited by 50% while the alternative pathway is blocked by almost 100%.

### SCIN is a Human-Specific Complement Inhibitor

The above data indicate that SCIN inhibits all three complement pathways. To evaluate whether SCIN is human specific, SCIN was tested in an alternative pathway assay where C5b-9 mediated lysis of rabbit erythrocytes is analyzed (Servaes et al., J. Immunol. Meth. 140: 93-100 (1991)). In human serum at 2-30%, rSCIN (10 μg/ml) strongly inhibited AP hemolytic activity (FIG. 3a). However, in the presence of mouse serum, rSCIN-mediated inhibition of hemolysis did not occur. Moreover, in the presence of rat, dog, sheep, guinea pig, goat and cow sera, rSCIN (10 μg/ml) was unable to inhibit the AP at all tested serum concentrations (20% serum mediated lysis is shown as a representative for all serum concentrations in FIG. 3b).

These data clearly show that SCIN exclusively inhibited alternative pathway activation in humans and therefore is a human-specific complement inhibitor. This and the fact that the gene for SCIN is only found in staphylococci isolated from humans strongly indicate that SCIN particularly evolved in staphylococci that infect humans.

To study whether SCIN is produced during staphylococcal infections, sera from 80 healthy individuals and 20 patients suffering from recurrent staphylococcal infections were tested for antibodies against SCIN by ELISA. All individuals produced antibodies against SCIN, indicating that staphylococci actively produce SCIN in humans (FIG. 3c). The fact that antibody concentrations in patients were comparable to healthy donors is not surprising, since all tested healthy donors and patients also produced antibodies against staphylococcal crude cell walls indicating that all people have encountered staphylococci during their lives. Although the human-specificity of SCIN limits the study of this immune evasion molecule in animal models, these findings strongly implicate that SCIN is important for staphylococcal survival in the human host.

Using an ex vivo inflammation model it could be shown that SCIN also blocks phagocytosis of S. aureus in human whole blood (FIG. 3d).

### SCIN is Surface-Dependent

To understand how SCIN interferes with human complement, it was studied whether SCIN could directly bind complement components. Recombinant SCIN did not directly bind to purified C1s, rMASP-1, rMASP-2, C2, C3, C4, fD, fB or properdin as determined by ELISA, immune-precipitation, size exclusion chromatography and BIACORE. Analysis of rSCIN binding to foreign particles revealed that rSCIN also failed to bind zymosan or S. aureus directly (FIG. 4a). However, in the presence of human sera, rSCIN could bind to zymosan or S. aureus. Because binding occurred only at 37° C. (FIG. 4), SCIN binds particles during complement activation. Also, SCIN molecules present in staphylococcal supernatants bound to the bacterial surface during opsonization (FIG. 4b). At 10% serum, this binding reached a maximum within five minutes. Thus, as an excreted protein, SCIN binds to the bacterial surface during complement activation. These data show that SCIN is a surface-dependent complement inhibitor.

### SCIN Acts on C3 Convertases

The above experiments show that SCIN, as a relatively small protein, can inhibit all complement routes. It was thus hypothesized that SCIN either acts on C3, which functions in all complement pathways, or on separate molecules that have a similar function. An interaction with C3 or C3b was excluded since SCIN did not directly bind purified C3, C3b or surface-bound C3b.

The fact that C4b deposition in the CP and LP was not inhibited by SCIN proved that C1 and MBL-MASP complexes retained their activity (FIGS. 2g,h). Thus, SCIN affects the CP and LP downstream of C4b, but upstream of C3b deposition. This implies that SCIN may either interfere with the formation and/or the activity of the C4b2a convertase. Since C3 convertases of the CP and LP are functionally and structurally similar to those of the AP, a similar mechanism could apply for the AP.

To study the formation of the CP, LP and AP C3 convertases, the cleavage of C2 and fB was analyzed. When C2 and fB are in complex with particle-bound C4b and C3b respectively, they are cleaved by specific proteases. The C2b and Ba split products are released immediately after cleavage. The active convertase components C2a and Bb initially stay bound to the surface, but will be released eventually as a result of convertase instability. When zymosan was incubated with 10% serum, both released C2a and C2b could be detected after 20 minutes of incubation (FIGS. 5a,b). For the AP, Ba and Bb were detected in 5, 10 or 20% serum after 20 minutes of incubation with S. aureus (FIG. 5c).

In the presence of rSCIN (10 μg/ml), no C2 (FIGS. 5a,b) and fB (FIG. 5c) cleavage products were observed. These data show that SCIN prevents efficient cleavage of C2 and fB, and thus affects formation of C4b2a and C3bBb.

Formation of C4b2a and C3bBb is dependent on two steps: firstly, C2 and fB bind the surface-bound cofactors C4b and C3b; then C2 and fB are cleaved by C1s, MASP-2 or fD respectively. To examine the exact effect of SCIN on C3 convertase formation, it was studied whether the inhibitory actions of SCIN are dependent on surface-bound cofactors. In the absence of surface-bound C4b, the cleavage of purified C2 by C1s or recombinant MASP-2 (rMASP-2) was not affected by rSCIN at 10 μg/ml (FIGS. 5d,e). Also, the cleavage of fB in the absence of surface-bound C3b was not affected by rSCIN (10 μg/ml) (FIG. 5f). Because fD cleavage of fB only occurs in the presence of a cofactor, fluid-phase C3 (H₂O), the hydrolyzed form of C3 which also acts as a cofactor was used. However, in the presence of pre-opsonized particles that contain C3b, rSCIN (3 μg/ml) efficiently prevented fB-cleavage by fD (FIG. 5g). Thus, SCIN inhibits the generation of C3 convertases in the presence of an activator surface.

This surface-specificity of SCIN triggered the inventors to analyze C2 and fB molecules on the surface of opsonized particles. In the absence of SCIN, zymosan or bacteria did not contain detectable amounts of surface-bound C2, fB or one of their split products after 20 minutes of opsonization (FIGS. 6a,b). However, when opsonization was performed in the presence of rSCIN (10 μg/ml), large numbers of surface-bound C2a and Bb molecules were detected but not the smaller C2b or Ba molecules (FIGS. 6a and 6b). When zymosan was incubated in 20% serum, maximum binding of C2a by SCIN was reached within 10 minutes while Bb binding was maximal within 20 minutes (FIGS. 6c and 6d). Thus, SCIN stabilizes surface-bound C4b2a and C3bBb. Because C2a and Bb continuously dissociate from the convertase complex, surface detection of C2a and Bb underestimates the total amount of generated convertases. For a quantitative analysis of surface-bound C3 convertases, C3bBb complexes were stabilized on zymosan by adding properdin and Ni⁺⁺ to 5% human serum (Fishelson et al., J. Immunol. 129: 2603-2607 (1982)). Under these conditions, lower amounts of Bb were detected on zymosan in the presence of SCIN (FIG. 6e) indicating that SCIN indeed prevents convertase formation.

In summary, SCIN acts on both C4b2a and C3bBb, which explains why SCIN inhibits all complement pathways. On the surface of zymosan or S. aureus, SCIN causes stabilization of C3 convertases and prevents their dissociation (FIG. 6). Moreover, in supernatants of opsonized zymosan or S. aureus it was found that SCIN prevents cleavage of C2 and fB (FIGS. 5a,b), indicating that the observed stabilization of C3 convertases hinders the formation of additional convertases.

### SCIN Binds and Inactivates C3bBb

Next it was studied how SCIN interacts with activator-bound C3 convertases. Zymosan was pre-incubated with 20% human serum for 30 minutes to deposit C3b. These particles did not contain Bb or C2a (FIGS. 6c,d). SCIN did not bind pre-opsonized zymosan indicating that SCIN does not bind activator-bound C3b directly (FIG. 7a (−)).

Incubation of pre-opsonized zymosan with both fB and fD resulted in binding of SCIN (FIG. 7a (B+D)), while no binding occurred with fB or fD alone (FIG. 7a (B), (D)). This binding was dependent on the concentration of fD (FIG. 7b). So, SCIN binds zym-C3b in the presence of fB, but fB-cleavage is essential for SCIN binding. When zym-C3b was mixed with fB and fD in the presence of properdin and NiCl₂), stable C3bB and C3bBb complexes were created at the surface (FIG. 7c). After washing the particles, SCIN could efficiently bind to zym-C3bBb, but not zym-C3bB (FIG. 7d). These data clearly show that SCIN binds the AP C3 convertase C3bBb. SCIN cannot bind C3bB, but binds the convertases directly after fB is cleaved by fD.

Because C3bBb is responsible for cleavage of C3, it was studied whether SCIN-binding to C3bBb affects convertase activity. Recombinant SCIN (10 μg/ml) inhibited C3b deposition by C3bBb on zymosan (FIG. 7e). Therefore SCIN binding to C3bBb leads to catalytic inactivation of the C3 and C5 convertases and thereby results in decreased C3b deposition (see FIG. 2d-f) and C5b-9 deposition (see FIG. 2a-c).

### Example 2

### Homologues of SCIN

### Introduction

This example describes three molecules in S. aureus that are highly homologous to SCIN: SCIN-B, SCIN-C and ORF-D share 46%, 48% and 33% sequence similarity with SCIN. Although ORF-D had no effect on complement activation and phagocytosis, SCIN-B and SCIN-C proved to be potent complement inhibitors as well. In contrast to SCIN, SCIN-B and SCIN-C exclusively inhibited the alternative pathway. Although SCIN-B and SCIN-C stabilized C4b2a at the bacterial surface they could not prevent C3b deposition via the classical and lectin pathway both in ELISA and on S. aureus. However, SCIN-B and SCIN-C prevented phagocytosis and C5a production with an equal efficacy as SCIN.

### Methods

### SCIN Homologues

Cloning and expression of SCIN-B, SCIN-C and ORF-D was carried out as described for SCIN with use of the following primers: 5′-AGTAGTCTGGACAAATATTT-3′ (forward) and 5′-CCGGAATTCTTATCTATTTATAATTTCAT-3′ (reversed, containing EcoRI site (underlined)) for SCIN-B, 5′-GAGTAGTAAGAAAGACTATAT-3′ (forward) and 5′-GGAATTCCTTATCTATTTATAATTTCA-3′ (reversed) for SCIN-C and 5′-GAGCAAATCTGAAACTACATCACAT-3′ (forward) and 5′-GGAATTCCTTAATTGTTTTGTGAATGC-3′ (reversed) for ORF-D. Recombinant proteins were purified.

Phagocytosis and Complement Activation on S. aureus

Laboratory strain S. aureus Cowan EMS was used for phagocytosis and complement activation assays. Phagocytosis and C3b deposition experiments were performed as described above. Briefly, phagocytosis was performed by incubation of FITC-labeled bacteria with human sera and freshly isolated neutrophils for 15 minutes at 37° C. Reaction were stopped by 1% paraformaldehyde and bacterial uptake by 10.000 gated neutrophils was analyzed by flow cytometry. For C3b deposition, heat-killed S. aureus was incubated with 10% human sera for 30 minutes at 37° C. and FITC labeled anti-C3 antibodies were used to detect C3b/iC3b on the surface. Factor D deficient serum was prepared by size exclusion chromatography and tested in the AP50. To study C5a generation, supernatants after opsonization were used as a stimulus in a calcium flux assay with Fluo-4-AM labeled neutrophils.

### Complement Assays

Functional activity of the CP, LP and AP was screened as described above. Sera were pre-incubated with rSCIN, rSCIN-B or rSCIN-C for 15 min at room temperature. Alternative pathway hemolytic assay was performed by incubation of 2×10⁷ rabbit erythrocytes (Biotrading, Wilnis, The Netherlands) with serum in Veronal buffered Saline (VBS) containing 5 mM MgCl₂ and 10 mM EGTA for 1 h at 37° C. Cells were spun down and A₄₀₅ measurements of supernatants were taken.

### Analysis of C2 and fB

S. aureus Cowan EMS (5×10⁶) was incubated with serum for 20 min at 37° C. in HBS⁺⁺ or in HBS− 2 mM MgCl₂-2 mM EGTA respectively. Then, after centrifugation both the supernate and particle-associated proteins were subjected to SDS-PAGE and analyzed by immunoblotting. Human C2 and fB were detected with goat anti-human C2 (Quidel Corporation, San Diego, Calif.) or goat-anti human factor B (Merck, Darmstadt, Germany), followed by peroxidase-conjugated anti-goat IgG (Santa Cruz Biotechnology, Santa Cruz, Calif.).

### Statistical Analysis

Statistics were performed using the Student's T-test; P values of <0.05 were regarded as significant.

### Accession Numbers

The accession numbers for SCIN homologues in S. aureus are: SCIN-B: gi|13700958|dbj|BAB42254.1|, SCIN-C: gnl|Sanger 159288|Staphylococcus (from the Sanger database (S. aureus 252, (MRSA 16) and ORF-D: gi|21203369|dbj|BAB94070.1|.).

### Results

Identification of Three SCIN-Like Molecules in S. aureus

A BLAST search revealed that there are three SCIN-like molecules in S. aureus. These formerly unknown proteins share 46% (SCIN-B), 48% (SCIN-C) and 33% (ORF-D) sequence homology with SCIN. No significant homologues were found in other microorganisms. PCR analysis revealed that the genes for SCIN-B (scn-b), SCIN-C (scn-c) and ORF-D (orf-d) are carried by 68%, 54% and 98% of clinical S. aureus strains respectively. The scn-b, scn-c and orf-d genes all code for extracellular proteins containing a classical signal peptide that is cleaved off at the bacterial membrane. The excreted SCIN-B, SCIN-C and ORF-D are 85, 85 and 86 amino acids long and have a molecular weight of 9.8 kD. To study their role in staphylococcal immune evasion, the SCIN homologues were cloned and expressed in E. coli and subsequently purified to homogeneity as described above.

SCIN-B and SCIN-C Block Complement Activation on S. aureus

To test whether the SCIN-like molecules function as staphylococcal immune modulators, the recombinant proteins were first analysed in a phagocytosis assay. Although recombinant ORF-D (rORF-D) did not affect phagocytosis, recombinant SCIN-B and SCIN-C (rSCIN-B and rSCIN-C) efficiently prevented staphylococcal uptake by human neutrophils (FIG. 8a). As was observed earlier for SCIN, rSCIN-B and rSCIN-C strongly inhibited phagocytosis in serum concentrations of 3% and higher, but not at lower serum concentrations. In 10% human sera the IC₅₀ was 0.3 μg/ml for rSCIN, 0.8 μg/ml for rSCIN-B and 0.12 μg/ml for rSCIN-C (FIG. 8a). Combining different SCIN molecules resulted in an additional rather than a synergistic effect.

To study the effect of SCIN-like molecules on complement activation, both deposition of C3b and the release of the anaphylatoxin C5a after opsonization of staphylococci were analysed. Using anti-C3 antibodies it was shown that rSCIN-C blocked C3b deposition on S. aureus with an efficacy equal to rSCIN. Recombinant SCIN-B was less efficient in preventing opsonization of staphylococci (FIG. 8b).

Furthermore, it was observed that rSCIN-B and rSCIN-C effectively prevented release of C5a during opsonization (FIG. 8c). Since rORF-D could not block phagocytosis, C3b deposition or C5a production, this 33% homologue to SCIN is not a staphylococcal complement inhibitor (FIG. 1a-c). SCIN-B and SCIN-C are both staphylococcal complement inhibitors with comparable anti-opsonic activity as SCIN.

### SCIN-B and SCIN-C Strongly Inhibit the Alternative Pathway

SCIN completely blocks AP-mediated C3b deposition while the CP and LP are inhibited by 50%, as was shown by pathway-specific complement ELISA's. Therefore, the pathway-specificity of rSCIN-B and rSCIN-C was also tested. Both rSCIN-B and rSCIN-C proved to be very effective inhibitors of the AP (FIG. 9a). However, rSCIN-B and rSCIN-C did not prevent CP-mediated C3b deposition (FIG. 9b), and the LP was weakly inhibited at 1% serum (FIG. 9c). On S. aureus it was also observed that SCIN-B and SCIN-C exclusively prevented C3b deposition via the AP (FIGS. 10a,b). In the presence of Mg-EGTA, SCIN-B and SCIN-C prevented C3b deposition with an equal efficacy as SCIN (FIG. 10a). However, C3b deposition in factor D-deficient serum was not inhibited by SCIN-B and SCIN-C (FIG. 10b). In conjunction with the ELISA, SCIN prevented C3b deposition via the CP/LP by 50% (FIG. 10b). In Example 1 it was described that SCIN is highly human-specific. Also, SCIN-B and SCIN-C solely inhibited AP-mediated hemolysis of rabbit erythrocytes in human sera but not in sera of other tested animals (mouse, rat, dog, sheep, guinea pig, goat, and cow). In summary, SCIN-B and SCIN-C are very potent inhibitors of the AP, but do not strongly inhibit the CP and LP in ELISA.

SCIN-B and SCIN-C Affect C4b2a and C3bBb

Since SCIN is described to stabilize C3 convertases on microbial surfaces, the effects of SCIN-B and SCIN-C on C3 convertases were analysed. First of all it was observed that rSCIN-B and rSCIN-C also stabilized both C3 convertases on the staphylococcal surface. High amounts of Bb and C2a could be detected on the surface after opsonization in the presence of rSCIN, rSCIN-B and rSCIN-C (FIGS. 11a, b). Furthermore, the generation of the total amount of C4b2a and C3bBb was also inhibited by rSCIN-B and rSCIN-C (FIGS. 11c, d). Formation of C4b2a was most efficiently prevented by rSCIN and rSCIN-C. Altogether it can be concluded that the effect of SCIN-B and SCIN-C on C3bBb is similar to SCIN. Moreover, SCIN-B and SCIN-C are capable of stabilizing and preventing formation of C4b2a.

### CONCLUSION

In this Example it is demonstrated that SCIN belongs to a larger family of staphylococcal complement inhibitors. Next to SCIN, two homologous complement inhibitors were identified, SCIN-B and SCIN-C sharing 46% and 48% homology. The third SCIN homologue (33%), ORF-D, did not function as a complement modulator in our assays. In contrast to SCIN, SCIN-B and SCIN-C were shown to be specific inhibitors of the AP and did not interfere with CP and LP.

1.-21. (canceled)

22. A method for treating, diagnosing, or preventing an indication that involves complement activation via the classical and/or alternative pathway comprising:
administering to a patient in need thereof, a therapeutically acceptable amount of staphylococcal complement inhibitor (SCIN) or a homologue thereof, or a derivative or a fragment of SCIN or the SCIN homologue, or mixtures thereof.

23. The method of claim 22, wherein the indication that involves complement activation via the classical and/or alternative pathway are treated via intervening with C3 and C5 convertases.

24. The method of claim 22, wherein the indication that involves complement activation via the classical and/or alternative pathway are treated via inhibiting activation of the classical and/or the alternative pathway of complement.

25. The method of claim 22, wherein the indication that involves complement activation via the classical and/or alternative pathway are inflammatory reactions.

26. The method of claim 22, wherein the staphylococcal complement inhibitor (SCIN) is encoded by an isolated nucleic acid molecule comprising an nucleotide sequence corresponding to a sequence selected from the group consisting of:
a) a nucleotide sequence comprising a part of one of a sequences of SEQ ID NO. 9; SEQ ID NO: 10; SEQ ID NO: 11; or SEQ ID NO:12;
b) a nucleotide sequence which encodes the amino acid sequence of SEQ ID NO:15; SEQ ID NO: 16; SEQ ID NO:17; or SEQ ID NO:18;
c) a nucleotide sequence which encodes a portion of the amino acid sequence of SEQ ID NO:15; SEQ ID NO: 16; SEQ ID NO:17, or SEQ ID NO:18;
d) a nucleotide sequence at least 35% to the nucleotide sequences of a), b) or c);
e) a nucleotide sequence hybridized at stringent conditions made with any one of the nucleotide sequences of a), b), c) or d);
f) a nucleotide sequence complementary to any of the nucleotide sequences of a), b), c), d), or e).

27. The method of claim 26, wherein the nucleotide sequence corresponds to nucleotides 1-490 of SEQ ID NO:9 or SEQ ID NO: 10.

28. The method of claim 26, wherein the nucleotide sequence corresponds to nucleotides 41-490 of SEQ ID NO:9 or SEQ ID NO: 10.

29. The method of claim 26, wherein the nucleotide sequence corresponds to nucleotides 125-490 of SEQ ID NO:9 or SEQ ID NO: 10.

30. The method of claim 26, wherein the nucleotide sequence corresponds to nucleotides 13-360 of SCIN-B (SEQ ID NO:11) or SCIN-C (SEQ ID NO:12).

31. The method of claim 26, wherein the nucleotide sequence corresponds to nucleotides 106-360 of SCIN-B (SEQ ID NO:11) or SCIN-C (SEQ ID NO:12).

32. The method of claim 26, wherein the nucleotide sequence is at least 40% identical to any one of the nucleotide sequences of SEQ ID NO:9; SEQ ID NO:10; SEQ ID NO:11; or SEQ ID NO:12; or the nucleotide sequences which encode the amino acid sequences of SEQ ID NO:15; SEQ ID NO:16; SEQ ID NO:17 or SEQ ID NO:18.

33. The method of claim 26, wherein the stringent conditions are constituted by overnight hybridization at 42° C. in 5×SSC and washing at 65° C. at 0.1×SSC.

34. The method of claim 26, wherein the amino acid sequence which encodes the nucleotide sequence constitutes alone or with other portions of the amino acid sequence the region(s) of the polypeptide having SCIN activity that leads to biological activity.

35. The method of claim 22, wherein the SCIN homologue is SCIN-B or SCIN-C.

36. The method of claim 22, wherein the method is for diagnosing an indication that involves complement activation via the classical and/or alternative pathway.

37. The method of claim 22, wherein the method is for treating an indication that involves complement activation via the classical and/or alternative pathway.

38. The method of claim 22, wherein the method is for preventing an indication that involves complement activation via the classical and/or alternative pathway.

39. The method of claim 25, wherein the inflammatory reaction is acute and chronic.

40. The method of claim 25, wherein the method is for treatment of the inflammatory reaction is for treating any of the diseases listed in Table 2.

41. The method of claim 22, further comprising: administering in composition with a suitable excipient.

42. The method of claim 26, further comprising: administering via a micro-organism which harbors the nucleic acid molecule.

43. The method of claim 26, wherein the nucleotide sequence is at least 46% identical to any one of the nucleotide sequences of SEQ ID NO:9; SEQ ID NO:10; SEQ ID NO:11; or SEQ ID NO:12; or the nucleotide sequences which encode the amino acid sequences of SEQ ID NO:15; SEQ ID NO:16; SEQ ID NO:17 or SEQ ID NO:18.

44. The method of claim 26, wherein the nucleotide sequence is at least 48% identical to any one of the nucleotide sequences of SEQ ID NO:9; SEQ ID NO:10; SEQ ID NO:11; or SEQ ID NO:12; or the nucleotide sequences which encode the amino acid sequences of SEQ ID NO:15; SEQ ID NO:16; SEQ ID NO:17 or SEQ ID NO:18.

45. The method of claim 26, wherein the nucleotide sequence is at least 50% identical to any one of the nucleotide sequences of SEQ ID NO:9; SEQ ID NO:10; SEQ ID NO:11; or SEQ ID NO:12; or the nucleotide sequences which encode the amino acid sequences of SEQ ID NO:15; SEQ ID NO:16; SEQ ID NO:17 or SEQ ID NO:18.

46. The method of claim 26, wherein the nucleotide sequence is at least 60% identical to any one of the nucleotide sequences of SEQ ID NO:9; SEQ ID NO:10; SEQ ID NO:1; or SEQ ID NO:12; or the nucleotide sequences which encode the amino acid sequences of SEQ ID NO:15; SEQ ID NO:16; SEQ ID NO:17 or SEQ ID NO:18.

47. The method of claim 26, wherein the nucleotide sequence is at least 70% identical to any one of the nucleotide sequences of SEQ ID NO:9; SEQ ID NO:10; SEQ ID NO:11; or SEQ ID NO:12; or the nucleotide sequences which encode the amino acid sequences of SEQ ID NO:15; SEQ ID NO:16; SEQ ID NO:17 or SEQ ID NO:18.

48. The method of claim 26, wherein the nucleotide sequence is at least 75% identical to any one of the nucleotide sequences of SEQ ID NO:9; SEQ ID NO:10; SEQ ID NO:1; or SEQ ID NO:12; or the nucleotide sequences which encode the amino acid sequences of SEQ ID NO:15; SEQ ID NO:16; SEQ ID NO:17 or SEQ ID NO:18.

49. The method of claim 26, wherein the nucleotide sequence is at least 80% identical to any one of the nucleotide sequences of SEQ ID NO:9; SEQ ID NO:10; SEQ ID NO:11; or SEQ ID NO:12; or the nucleotide sequences which encode the amino acid sequences of SEQ ID NO:15; SEQ ID NO:16 SEQ ID NO:17 or SEQ ID NO:18.

50. The method of claim 26, wherein the nucleotide sequence is at least 90% identical to any one of the nucleotide sequences of SEQ ID NO:9; SEQ ID NO:10; SEQ ID NO:11; or SEQ ID NO:12; or the nucleotide sequences which encode the amino acid sequences of SEQ ID NO:15; SEQ ID NO:16; SEQ ID NO:17 or SEQ ID NO:18.
